(19) United States
(12) Patent Application Publication
(10) Pub. No.: US 2004/0072165 A1
(43) Pub. Date: Apr. 15, 2004

(54) Method for producing dna encoding polypeptides that are composed of several section, and for producing polypeptides by expressing the dna thus obtained

(76) Inventor: Florian Schauwecker, Berlin (DE)

Correspondence Address: PROCOPIO, CORY, HARGREAVES & SAVITCH LLP, 530 B STREET, SUITE 2100, SAN DIEGO, CA, 92101, US

(21) Appl. No.: 10/258,567

(22) PCT Filed: Apr. 25, 2001
(86) PCT No.: PCT/DE01/01578

(30) Foreign Application Priority Data
Apr. 26, 2000 (DE) 100 21 267.0

(51) Int. Cl. C12Q 1/68; C12P 19/34

(52) U.S. Cl. 435/6; 435/91.2

(57) ABSTRACT

The invention relates to a method for producing novel modular enzyme systems by genetic engineering, which is characterized by a cyclic in vitro gene synthesis and which allows a specific recombination of the individual, gene-encoded modular components to novel enzyme systems. Examples of such modular enzyme systems are the non-ribosomal peptide synthetases (NRPS) or polyketide synthases (PKS) of type 1, that is the amino acid sequence of such an enzyme is characterized by being composed of a repetitive sequence of identical sequence sections or sequence sections that are very similar to one another. Every single repetitive sequence is referred to as a module and every module allows the enzymatic incorporation of a specific substrate into the substance synthesized by the enzyme. The products synthesized by the NRPS and PKS enzymes are often highly valuable as pharmaceuticals, such as the penicillins, vancomycins or erythromycins. The inventive method allows for an effective production of novel genes for modular enzymes, and the gene expression of said novel genes allows for the production of novel substances.

The present invention relates to methods for the preparation of DNA which code for polypeptides which are composed of a plurality of sections, and of DNA vectors and cells which comprise this DNA, and to methods for the preparation of such polypeptides by expression of the DNA obtained in this way, and to the use of these polypeptides preferably as enzymes for the synthesis of novel product compounds with bioactive and/or pharmacologically active potential.

Certain classes of proteins have a structure of their polypeptide chain which is divided into more than one section, such as, for example, one divided into individual domains. This is the case in particular with proteins having a modular structure, where as a rule a plurality of different enzymatic activities or functions are combined on a single polypeptide chain.

The amino acid sequence of an enzyme having a modular structure is characterized in that it is composed of a repetitive sequence of sequence sections which are identical or structurally very similar to one another and which are referred to as modules. Important representatives of enzyme systems having a modular structure are non-ribosomal peptide synthetases (NRPS) and polyketide syntheses (PKS) of type 1. The products synthesized by the NRPS and PKS enzymes are often of high pharmaceutical value, such as, for example, the penicillins, vancomycins or erythromycins.

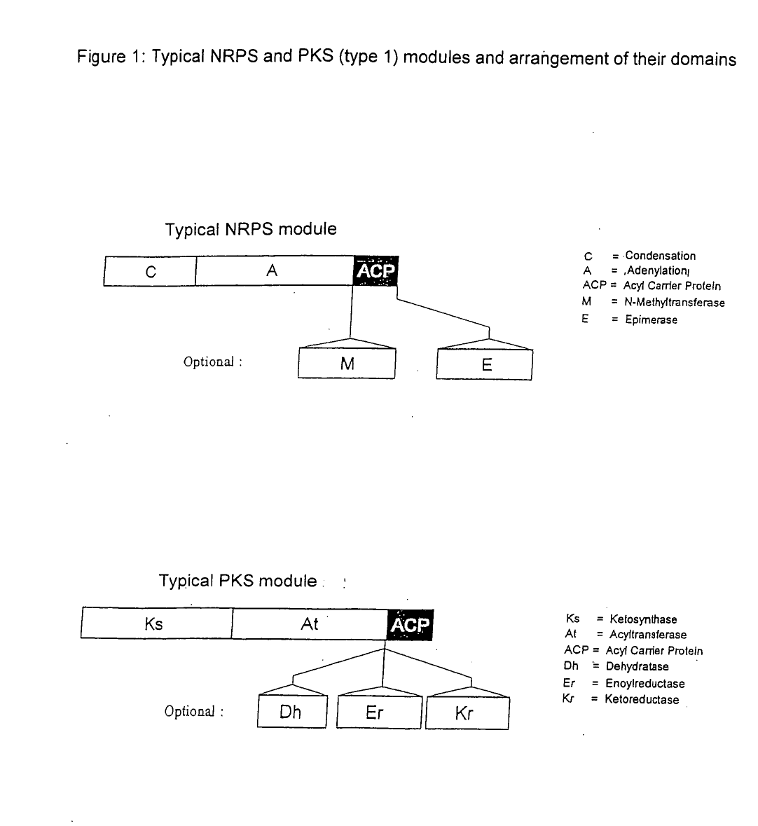

The structure and the function of peptide synthetases (NRPS) are known in the state of the art (1-4, R1). NRPS enzymes catalyze the synthesis of peptides from amino acids, and each module of an NRPS enzyme as a rule catalyzes the incorporation of exactly one amino acid into the resulting product. The difference between the individual modules is that they use different amino acids as substrates. It is also known that some modules are able to modify the amino acid used, such as, for example, by methylation or epimerization. All functional properties of an NRPS module can be assigned to particular subregions of the module which are referred to as domains. The presence of a domain for recognition and adenylation of the substrate amino acid used (adenylation domain), of a domain for covalent bonding of the substrate amino acid (ACP domain) and of a domain for linkage of the substrate amino acid to-the substrate of an adjacent module (condensation domain) is essential for an NRPS module. Domains for substrate modification, such as a domain for N-methylation of the substrate amino acid (methylation domain) or a domain for epimerization of the substrate amino acid; are optional (FIG. 1).

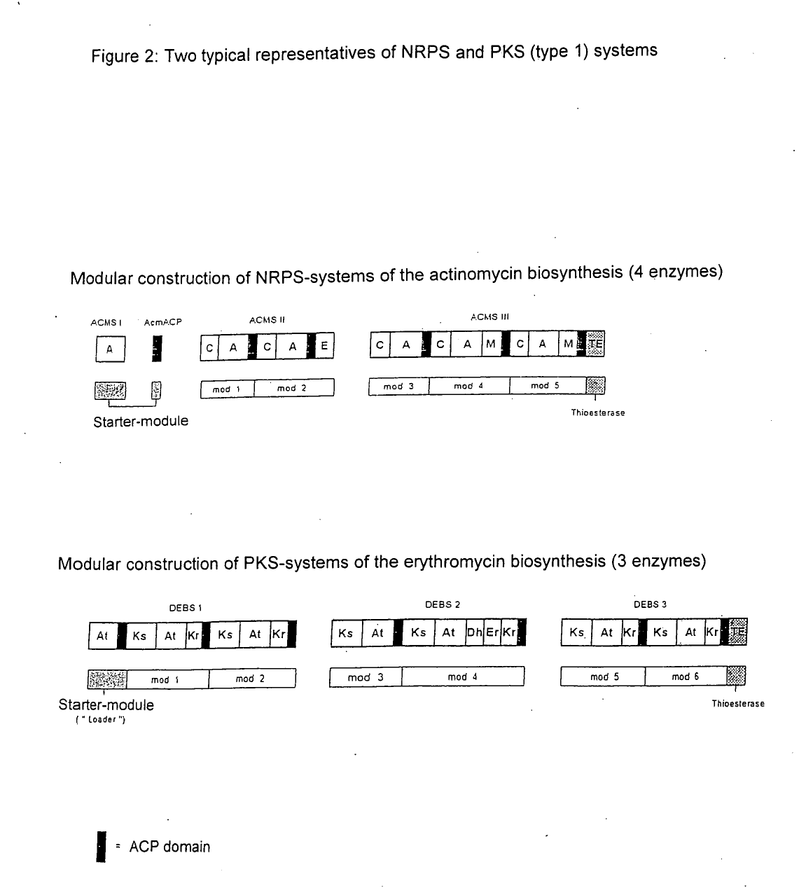

The structure and the function of polyketide synthases (PKS) of type 1 are likewise known in the state of the art (5-11, R2, R3). PKS enzymes catalyze the formation of polyketides which can be described in simple terms as a concatenation of C₂ units. The PKS enzymes use as substrates coenzyme A (CoA)-activated carboxylic acid derivatives such as malonyl-CoA, methyl-malonyl-CoA or ethylmalonyl-CoA, from which the C2 units used for the synthesis are derived. In a similar way- to the NRPS enzymes, the modules of PKS enzymes are also composed of distinct domains with specific functions. Such a PKS module has at least one domain for recognition of the substrate (AT domain), one domain for covalent bonding of the substrate (ACP domain), and one domain for linkage of the building block used to the building block of an adjacent module (KS domain). There are also optional domains in PKS modules, such as a domain for reduction of keto groups (KR domain), a domain for dehydrogenation (DH domain), or a domain for reduction of unsaturated carbon-carbon bonds (ER domain) (FIG. 2).

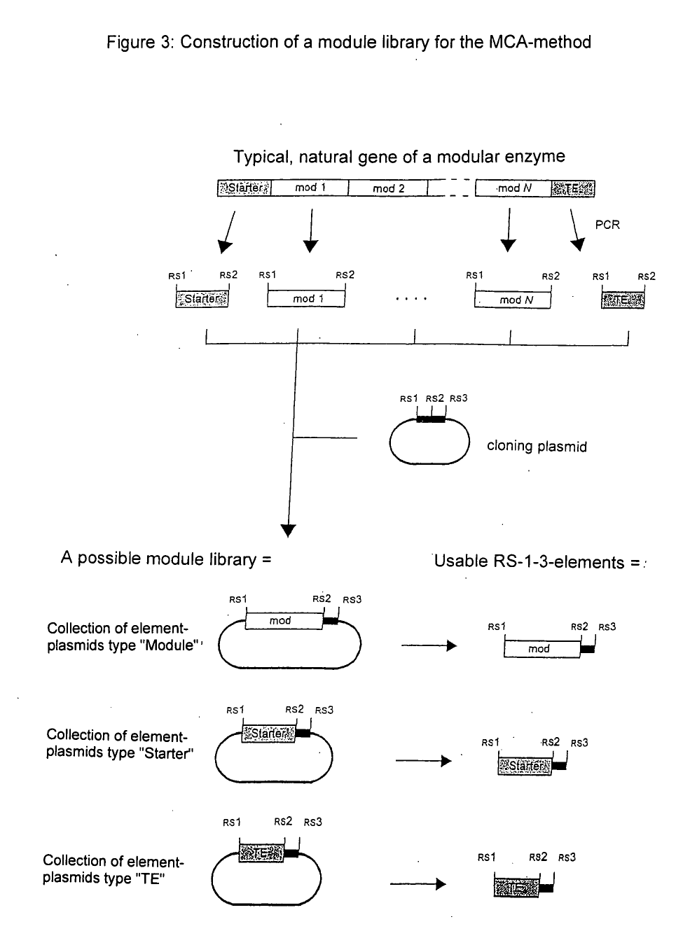

It is common to NRPS and PKS enzymes that the sequence of synthetic building blocks incorporated by them into the synthesized product is as a rule fixed directly by the sequence of their modules. There are also in both classes of enzymes synthesis systems in which the modules or domains are distributed over a plurality of enzymes which are present separately. Examples thereof are the PKS system of erythromycin biosynthesis (DEBS enzymes 1, 2 and 3) and the NRPS system of actinomycin biosynthesis (ACMS enzymes I, II, III and AcmACP) (FIG. 3).

Both NRPS and PKS enzymes may, besides the standard modules already described, also have modules with special domains or a different arrangement of the domains. Different modules of this type at the start of a PKS enzyme are generally referred to as loader module and make it possible for synthesis to start with unusual synthetic building blocks such as, for example, acetyl-CoA or propionyl-CoA. The individual domains of loader modules may also possibly be present as separate enzyme components. Some NRPS enzymes are likewise able to use unusual synthetic building blocks such as, for example, aromatic carboxylic acid derivatives to start the synthesis. In analogy to the loader modules of PKS enzymes, it is likewise possible with these NRPS enzymes for there to be a different arrangement of domains or the presence of unusual domains at the start of the enzyme. It is likewise possible for these domains to be present as separate enzyme components, such as, for example, in the actinomycin biosynthesis system.

With some NRPS enzymes, a start of the synthesis with unusual synthetic building blocks such as, for example, with fatty acid derivatives can also be made possible by a condensation domain located at the start of the enzyme. The release and/or cyclization of the synthesized product of many NRPS and PKS enzymes is catalyzed by a so-called thioesterase domain (TE domain) which is, as a rule, an integral constituent of the enzyme.

It is already known in the state of the art that exchange, insertion, deletion or rearrangement (referred to hereinafter inclusively as recombination) of modules or domains is possible and may lead to enzymes with altered specificity. Thus, modules or domains both in NRPS enzymes and in PKS enzymes have been exchanged and recombined by genetic manipulation (1-11). Some of these recombinant novel enzymes have been characterized in vitro or in vivo, and the production of novel substances has been unambiguously demonstrated.

Recombination of modules or domains of enzymes having a modular structure, such as the polyketide synthases or the non-ribosomal peptide synthetases, have thus in principle introduced the possibility of preparing non-naturally occurring enzymes of these classes. The novel enzymes obtained in this way can be employed for synthesizing novel compounds.

Naturally occurring modular enzymes such as, for example, the PKS enzymes in streptomyces in many cases catalyze the formation of secondary metabolites which are of great pharmacological interest and have been introduced into a large number of therapeutic procedures. However, it is increasingly being found that disease organisms develop resistance to the active ingredients employed, for example, as antibiotics and thus there is still a very great need for novel compounds having appropriate pharmacological activity in each case. Such compounds are frequently identified by testing the compounds of a substance library in a high-throughput method. On the other hand, previously known pharmacologically active compounds can have their chemical structure modified in order in this way to increase the activity or circumvent a resistance which has already developed.

It is known in the state of the art that it is possible in principle by recombination of modules, domains or else only certain sections of an enzyme having a modular structure to produce certain modified natural substances via the novel enzyme obtained in this way. This has, nevertheless, been shown to date only for a few enzyme systems. The DNA coding for these novel enzymes has been obtained in each case by carrying out cloning steps tailored to the specific individual case.

It is, however, clear that a very large number of novel enzymes is necessary in particular for producing a comprehensive substance library with completely novel members, and a simple, standardized method for synthesizing the DNA coding for these novel enzymes is absolutely necessary for this.

As already mentioned, the only methods described in the state of the art are those in which clonings tailored to the specific case are carried out. In particular, these methods entail the DNA fragments coding for modules, domains or other sections of an enzyme having a modular structure being linked together via restriction cleavage sites which have in each case been selected for the specific individual case. However, this means that standardization of such methods is precluded.

In methods described in the state of the art [12] which attempt recombination of modular systems via in vivo combinatorial chemistry, the module introduced, and thus the module arrangement resulting and the nature of the fusion points, cannot be predicted; on the contrary, possible recombination tends to take place on a random basis.

It is therefore an object of the present invention to provide a method with which it is possible in a simple and standardized way to prepare polypeptides which are composed, like, for example, modular enzyme systems, from a plurality of sections, the structure of the latter optionally being fixed beforehand or, alternatively, a library of different polypeptides being created by a combinatorial approach.

The invention firstly relates to a method for preparing the DNA which codes for a polypeptide composed of a plurality of sections by linking together individual DNA fragments which code for the respective sections of such a polypeptide, and the polypeptides themselves are prepared in a subsequent method by expression of the DNA obtained in this way. Thus, one aspect of the invention is a method for preparing a DNA which codes for a polypeptide composed of a plurality of sections in a circular DNA vector, which method comprises the following steps:

(a) restriction of unique restriction cleavage sites RS1 and RS3 or of unique restriction cleavage sites RS2 and RS3 of the DNA vector;

(b) ligation of a DNA fragment which codes for at least one section of the polypeptide into the DNA vector obtained in (a),

i. where one end of the DNA fragment has been obtained by restriction of a restriction cleavage site RS1 and the second end of the DNA fragment has been obtained by restriction of a restriction cleavage site RS3, and the DNA fragment has no internal restriction cleavage site RS1 or internal restriction cleavage site RS3,

ii. where the restriction cleavage site RS1 is located at the 5′ end of the region (AKB) coding for the at least one section, and the restriction cleavage site RS3 is located at the 3′ end of the AKB,

iii. where the DNA fragment has a unique restriction cleavage site RS2 which is located between the restriction cleavage sites RS1 and RS3, and

iv. where the ends generated by restriction of the restriction cleavage sites RS1 and RS2 are compatible with one another, the DNA sequence resulting from the ligation is different from the restriction cleavage sites RS2 and RS3, and the ends generated by restriction of the restriction cleavage sites RS2 and RS3 are not compatible with one another;

(c) restriction of the unique restriction cleavage sites RS2 and RS3 in the DNA vector obtained in (b);

(d) ligation of another DNA fragment which codes for at least one other section of the polypeptide into the DNA vector obtained in (c), where the AKBs form a continuous reading frame and where the conditions defined in b) i.) to iv.) are to be applied appropriately, and, if desired,

(e) at least one repetition of steps c) and (d).

The polypeptides composed of a plurality of sections are preferably modular enzyme systems such as, for example, non-ribosomal peptide synthetases, polyketide synthases or receptors with a modular structure. A section of a polypeptide may be a complete module of such an enzyme system. Thus, in a non-ribosomal peptide synthetase, the section of the polypeptide chain which is meant as the one which catalyzes the incorporation of exactly one amino acid into the resulting module. However, the section of a polypeptide may, for the purposes of the present invention, likewise be only part of a module, e.g. a domain of a module. Thus, the domains meant in the case of NRPS are those to each of which a particular catalytic property is to be assigned within a module, such as, for example, the ACP domain which catalyzes covalent bonding of the substrate amino acid, or the condensation domain of an NRPS enzyme system, which brings about linkage of the substrate amino acid of the relevant module to the substrate of an adjacent module. However, it must be remembered that a section of a polypeptide means for the purposes of the invention any subsection of a polypeptide chain without a particular function in relation to the overall function, such as, for examples the catalytic activity of a modular enzyme system, necessarily being assigned in every case to the section. Such sections of a polypeptide may be, for example, merely parts of a domain. The abbreviation “AKB” used herein (protein-encoding region for a section) of a DNA fragment relates to the part of a DNA which codes for a section of a polypeptide composed of a plurality of sections, where the term “section” has the meaning indicated above.

The method according to the invention can be used particularly advantageously for preparing DNA which codes for enzyme systems with a modular structure. This entails, for example, DNA fragments which code for complete module sections or part-sections of an enzyme system being assembled by DNA fusings, taking place cyclically in vitro, at defined fusion points to result in a continuous reading frame which is extended after each cycle and which codes for a complete modular enzyme or only a region of a modular enzyme, and being used for preparing such modular enzyme systems by gene expression.

The circularity of the DNA vector employed is a necessary condition for carrying out the method according to the invention. The DNA fragment to be inserted in each cycle of the method into the DNA vector requires the opening of the circular DNA vector as well as in each case the closing of the vector which terminates a cycle. If desired, the DNA vector obtained after a cycle and enlarged by one DNA fragment can be amplified after introduction into a suitable host organism and be prepared anew in sufficient quantity for use in the subsequent cycle of the method. An alternative possibility is to dispense with this amplification step if the DNA vector which results and contains a new DNA fragment is separated after each cycle, by methods known in the state of the art, from excess DNA fragments still present in the solution. The skilled person is moreover aware that the DNA fragment which is liberated by restriction during a cycle in step (a) and (c) in addition to the linear DNA vector fragment must be removed for the subsequent operations in the method, and the restriction enzymes employed for the abovementioned restriction in each case must. Likewise be removed or inactivated.

The techniques of molecular biology included in the individual steps of the method, such as restriction and ligation of DNA, are established standard methods in the state of the art. The location of the restriction cleavage site RS1 at the 5′ end and of the restriction cleavage site RS3 at the 3′ end of the region (AKB) coding for a section of a polypeptide includes both a location of the restriction cleavage sites flanking the AKB and a location of the restriction cleavage sites directly forming the boundary of the AKB. Directly adjacent means from this viewpoint that the DNA region surrounding the restriction cleavage site belongs directly to the AKB. By contrast, a flanking location of the restriction cleavage sites means that although the DNA region surrounding the restriction cleavage sites is protein-encoding because the creation of a continuously protein-encoding reading frame as a result of the linkage of the DNA fragments is absolutely necessary, the protein-encoding region of the restriction cleavage sites is not enclosed by the AKB.

The ends generated by restriction of the restriction cleavage sites RS1 and RS2 can be linked together by ligation because the corresponding ends are compatible with one another, although it is a condition that the respective restriction cleavage sites RS1 and RS2 are different, so that ligation of the protruding ends of the restriction cleavage sites RS1 and RS2 results in a DNA sequence section which has neither the RS1 nor the RS2 recognition site for restriction enzymes. The method according to the invention relates to the preparation of a DNA in a circular DNA vector which codes for a polypeptide consisting of at least two sections, with optional linkage, through repetition of the cycle, of another DNA fragment in each of the cycles with the DNA already present in the DNA vector.

An alternative embodiment of the invention relates to a method where the DNA which codes for at least one section and which is employed in stage (d) as second or later DNA fragment has been obtained by restriction of the restriction cleavage sites RS2 and RS3, and the DNA fragment has no internal restriction cleavage site RS2 or internal restriction cleavage site RS3, where the restriction cleavage site RS2 is located at the 5′ end of the AKB and the restriction cleavage site RS3 is located at the 3′ end of the AKB, and this DNA fragment is thus employed as the fragment concluding the cycle.

No special termination step is necessary to conclude the method of the invention for producing a polypeptide composed of a plurality of sections, although the specific location and compatibility or noncompatibility of cleaved restriction cleavage sites in this method makes it possible also to use a DNA fragment that has the ends generated by restriction of the restriction cleavage sites RS2 and RS3 for termination of the method.

In another embodiment of the invention there is use in the method of at least one DNA fragment which comprises a DNA which-codes for a section and which has at least one mutation compared with the naturally occurring nucleic acid sequence. A naturally occurring nucleic acid sequence means in this connection the DNA sequence which is the basis for the above at least one DNA fragment and is present genomically or on an extrachromosomal element in a naturally occurring organism and has been obtained as DNA for example by a PCR method.

The method of the invention makes it possible to prepare novel enzymes by recombination or exchange or else deletion of modules, domains or, in general, sections of a polypeptide such as, for example, of an enzyme system having a modular structure. It is clear to the skilled worker that the maximum diversity of novel enzymes which is generated is determined by the number of DNA fragments which are capable of linkage and which code for individual sections of a polypeptide. The method of the invention makes it possible to use DNA fragments which have previously been subjected to directed or undirected mutagenesis, and in this way to obtain a very large number of novel enzymes. Mutation means in this connection any change in the DNA sequence of a DNA fragment to be cloned, which results in a change in the primary structure of the polypeptide for which the at least one DNA fragment codes. These mutations may have been introduced for example by site-directed mutagenesis such as, for example, oligonucleotide-directed mutagenesis, and by undirected mutagenesis. The mutagens, i.e. chemical agents or physical influences such as UV rays and high-energy radiation, preferably used for undirected mutagenesis are those suitable for inducing point mutations at the level of the DNA sequence. These point mutations relate to one or a few adjacent base pairs of a DNA sequence section. The skilled worker is aware that both deletions, insertions, substitutions and reading frame mutations are meant in this connection.

A DNA vector which already comprises a protein-encoding reading frame is preferably used in the method of the invention, with the cloning of a DNA fragment in the manner described previously resulting in this protein-encoding reading frame being extended by at least two AKBs in the same reading frame. It is particularly preferred in this connection for the protein-encoding reading frame already present in the DNA vector employed to comprise at least one AKB.

In the case of the synthesis of a DNA coding for a modular enzyme system such as, for example, an NRPS enzyme system, it is particularly advantageous for the DNA coding for the start of the enzyme for certain modules or domains, such as, for example, a DNA section coding for an initiation module, already to be present in the circular DNA vector employed because in this way, especially in a combinatorial approach to the synthesis of many DNA sequences, for example, the sequence section coding in each case for the start section of the polypeptide is identical in all DNAs. However, it must be remembered that the protein-encoding reading frame which is optionally already present in the DNA vector is not confined to single modules or single domains. Thus, for example, it is possible in the method to extend a DNA section coding for a modular enzyme system by further modules or domains or parts thereof. The extension of the protein-encoding reading frame can take place both at the 5′ end and at the 3′ end of the protein-encoding reading frame, with the appropriate pair of restriction cleavage sites RS1/RS3 or RS2/RS3 being located either at the 5′ end or at the 3′ end of the protein-encoding region and, in addition, linkage in the same reading frame in relation to the already existing protein-encoding reading frame being ensured.

It is possible and advantageous to use as circular DNA vector in the method a plasmid vector, a lambda vector, a cosmid vector, the replicative form of the genome of a filamentous phage, or an artificial chromosome.

The term circular DNA vectors means all DNA elements which can be used for incorporating foreign DNA into a host cell and are suitable for DNA amplification, the circularity condition expressing the fact that these DNA elements must not have a 5′ or 3′ end. They are accordingly independently replicating DNA units such as, for example, derivatives of plasmids, viral genomes or artificially produced minichromosomes, of yeast or of bacteria (YAC or BAC). It is not a necessary condition that these DNA units undergo permanent independent replication after introduction to a host organism. Thus, the term circular DNA vectors also covers DNA units which temporarily integrate into the genome of the host organism and thus temporarily do not replicate independently. However, it is absolutely necessary that these DNA units can be isolated in a circular form separate from the genome of the host organism.

It is possible and preferred to use stably replicating plasmids in the method of the invention. These plasmids comprise both bacterial plasmids and the few stably replicating plasmids in eukaryotic microorganisms, such as, for example, the 2-micron plasmid of yeast. Shuttle vectors are particularly advantageous and make it possible to transfer the cloned DNA into other organisms too. All DNA vectors which can be used in the method of the invention have an origin of replication, the pair of restriction cleavage sites RS1/RS3 or RS2/RS3. Further details of DNA vectors and of the recombinant DNA techniques mentioned herein can be found in the standard work by Sambrook, Fritsch and Maniatis, Molecular Cloning—a Laboratory Manual, New York: Cold Spring Harbour Laboratory Press 1989.

It is particularly advantageous to use a plasmid vector which stably replicates in at least one bacterium, preferably in. Escherichia coli and/or in at least one streptomyces, and for the plasmid vector to have a selection marker and a cloning site comprising at least the unique restriction cleavage sites RS1 and RS3 or the unique restriction cleavage sites RS2 and RS3.

The DNA fragments preferably employed in the method described above have been obtained by PCR amplification from the genome of at least one microorganism and/or at least one plant, particularly preferably from the genome of actinomyces, and the restriction cleavage sites RS1, RS2 and RS3 have been introduced by means of directed mutagenesis.

A large number of gene sequences which code, for example, for enzyme systems with a modular structure (NRPS and PKS enzyme systems or receptors with a modular structure) are known from various databases In addition, there are available in databases a virtually unlimited number of DNA sequences which code for particular functional domains of other proteins which do not necessarily have a modular structure. It is possible, by isolating corresponding genomic DNA or extrachromosomal elements from the organism harboring these genes, and synthesizing suitable primer sequences, to provide the DNA sequence sections of interest, by PCR amplification, in sufficient quantity for the method of the invention. The primers required are two synthetically prepared oligodeoxynucleotide sequence sections with a length of from 15 to 30 nucleotides, whose sequences are complementary to the initial and final sequences of the partner strands of the DNA to be amplified. The PCR established in the prior art permits doubling of the DNA in each cycle through the three reaction steps repeated in the cycle. Details of the PCR technique can be found in Junis et al., PCR Protocols, London: Academic Press 1990. Site-directed mutagensis is used to introduce the restriction cleavage sites RS1, RS2 and RS3, if not already present, into the DNA fragment employed in the method, with simultaneous deletion where appropriate of internal restriction cleavage sites RS1, RS2 and RS3 which are present, likewise by site-directed mutagenesis. Ordinarily, in vitro methods should be used for specific introduction of mutations (base pair alterations, insertions, deletions) into DNA for the introduction or deletion of the above restriction cleavage sites, although the skilled worker is aware that, for example, it is also possible in bacteria or in yeasts to introduce inserted DNA by in vivo recombination at sequence-homologous sites. In these cases, certain DNA sequence sections can be specifically inactivated by insertion, or novel DNA sequence sections can be integrated at particular sites. However, ordinarily, only alteration of single or a few bases or introduction of small insertions or deletions will be necessary, which is easily possible through the use of synthetic oligonucleotides. Mutagenesis then takes place either by PCR or by vectors in single-stranded DNA form. Further details of site-directed mutagenesis are known to the skilled worker or can be found in Methods Enzymol. 154, 350-567 (1987) and Nucleic Acids Res. 16, 69, 6987-6999 (1988).

In a particularly advantageous embodiment, it is possible to prepare simultaneously a plurality of DNA sequences which code correspondingly for a plurality of polypeptides in a combinatorial approach without additional method steps. For this purpose, a mixture of at least two DNA fragments containing different AKBs are employed in step (b) and/or step (d).

At least two different DNA sequences and, consequently, as the result of expression, two different polypeptides are obtained by employing at least two DNA fragments which differ in at least one cycle of the stepwise extension of a DNA sequence by linkage to a DNA fragment. It is thus possible, by standardizing the method of the invention, to prepare in a very simple manner and without additional effort in parallel a very large number of different DNA sequences which code, for example, for a large number of different enzyme systems with a modular structure. It is advantageous for the DNA fragments employed in each cycle to be used in the same ratio of amounts relative to one another, so as to ensure that the result is a uniform random distribution of the DNA fragments at one position in the resulting-DNA.

The invention additionally relates to the DNA or a large number of DNA, which is referred to hereinafter as DNA library, where such a DNA library is obtained as the result of use of the combinatorial approach described above.

The invention likewise relates to a cell which comprises at least one DNA prepared by the method of the invention. The term cell means here every biological cell of a prokaryote or eukaryote, it being possible for the DNA present therein and prepared by the method of the invention to be present in this cell in various ways. Thus, the DNA may be present in a DNA vector which itself is comprised by the cell as extrachromosomal element and undergoes stable and autonomous replication in this cell. On the other hand, all prokaryotic or eukaryotic cells in which this DNA is stably integrated into the genome of the host organism are equally meant. The cell itself may be part of a multicellular organism or represent a single-cell organism. The invention relates to all organisms having at least one cell in which a DNA prepared by the method of the invention is present.

The invention further relates to the use of at least one DNA prepared by the method of the invention and to the use of the DNA library obtained by the combinatorial approach for preparing polypeptides.

For this purpose, the DNA is introduced and expressed in a suitable host organism, and the resulting polypeptide is optionally isolated from the latter.

The invention additionally relates to a method for preparing polypeptides consisting of at least two sections,

(a) where the at least one DNA present according to the method of the invention in the DNA vector and obtained therefrom by restriction is cloned into an expression vector,

(b) where the expression vector has a promoter and a start codon and termination codon for the reading frame of the at least one DNA which codes for a polypeptide consisting of at least two sections,

(c) where the resulting expression vector is introduced into a host cell for expression of the cloned at least one DNA,

(d) where the expression vector undergoes stable autonomous replication in the host cell or is stably integrated into the genome of the host cell, and

(e) where the at least one DNA is expressed in the host cell and, if desired, the at least one expression product is isolated from the host cell.

The polypeptide for which the DNA of the invention codes can be prepared on the one hand by recloning the appropriate DNA in a suitable expression vector, or alternatively the expression takes place directly starting from the DNA vector in which the DNA is directly present after the method for preparation thereof. It may be advantageous, depending on the nature of the DNA vector originally employed in the method for preparing the DNA, for this DNA to be cloned into a suitable expression vector.

Expression vectors which are appropriate for the particular host organism in which expression of the cloned DNA is to take place are advantageous. Thus, particularly advantageous expression vectors are those comprising a regulatable promoter, because it is possible in this way to increase expression stepwise and thus where appropriate to respond to a toxicity of the expression product or of corresponding secondary products. Besides the selection of an expression vector having a promoter tailored to the specific host organism and the specific expression product, account must be taken in particular of the stability of the vector and the number of copies of the recombinant DNA in the host organism. A possible toxicity of the gene product for the host organism can be overcome by use of a vector whose expression is inducible (e.g. by insertion of a lac operator between promoter and the DNA to be expressed). Expression then takes place only in the presence of an inducer (e.g. by IPTG on use of the lactose operon), there being no expression of the gene product in the repressed state, and unimpaired growth of the host organism being possible. In the induced state there is then strong, transient expression, and the expression product can be isolated biochemically. Although prokaryotic expression vectors are preferred, it is clear to the skilled worker that eukaryotic expression vectors may be more advantageous in some circumstances, if the disadvantage of eukaryotic expression vectors which is caused by the lower growth rates of eukaryotic cells is compensated by other advantages. Details of the selection of suitable vector systems for expression of the DNA of the invention can be found in Rodriguez and Denhardt (Editors), Vectors, Boston: Butterworths 1988.

The introduction of the DNA into the host cell can take place in various ways. On use of a bacterial cell for expression, the DNA vector containing the DNA can be taken up as free DNA molecule from the surroundings of the host cell by transformation, or be introduced by conjugation, in which the DNA is transferred directly from one cell to another, or by bacteriophage-mediated transduction, where the DNA is inserted into the cell through the interaction of a bacteriophage with the bacterium (e.g. the F pili structure). It is clear to the skilled worker that it is possible, although less advantageous, to introduce the DNA without using a vector. Transforming vector systems exist, although less diverse, for use of a eukaryotic cell as host cell for the expression. If expression in a plant is desired, it is possible to have recourse for example to the conjugative plasmids of the Gram-negative bacterium Agrobacterium tumefaciens. A number of prior-art methods are likewise known for introducing DNA vector constructs into yeast cells. It is advantageous to select the host cell used for the expression such that it shows a high growth rate, tolerate strong expression of the polypeptide derived from the recombinant DNA and, where appropriate, are modified in such a way that, for example, the genes of certain proteolytic enzymes have been deleted.

An alternative embodiment of the invention relates to a method for preparing polypeptides consisting of at least two sections,

(a) where the at least one vector containing the DNA is introduced by the method of the invention into a host cell,

(b) where the at least one DNA vector has a promoter and a start codon and termination cddon for the reading frame of the DNA which codes for a polypeptide consisting of at least two sections,

(c) where the at least one DNA vector undergoes stable autonomous replication in the host cell or is stably integrated into the genome of the host cell, and

(d) where the DNA is expressed in the host cell and, if desired, the at least one expression product is isolated from the host cell.

It is advantageous to use a microorganism as host cell in the two aforementioned methods for preparing polypeptides consisting of at least two sections, and a bacterium of the genus Streptomyces, Bacillus or Escherichia is particularly advantageous for this purpose.

The invention further relates to a polypeptide which is composed of a plurality of sections and which has been obtained by one of the aforementioned methods. These polypeptides comprise, for example, novel NRPS and novel PKS enzyme systems and novel hybrid NRPS-PKS enzyme systems.

The invention likewise relates to the use of a polypeptide obtained in the described manner for catalytic action on a compound or a mixture of a plurality of compounds. Thus, for example, hybrid NRPS enzyme systems claimed by the method of the invention can be used to synthesize novel peptides. The sequence of the amino acids in the peptide obtained in this way can be predetermined by specifying a particular linkage of sections of an NRPS enzyme system or, in the case of a large number of NRPS enzymes obtained by the previously described combinatorial approach, it is possible in this way to obtain a series of peptides whose structure is still unknown.

Another aspect of the invention is directed to a product compound which is obtained as a result of the catalytic action of the polypeptide on a precursor compound or a mixture thereof. Thus, for example, novel peptides comprising in particular non-proteinogenic amino acids can be obtained through the catalytic action of non-ribosomal peptide synthetases. These peptides may moreover, depending on the NRPS enzyme system used, be linear or cyclic. In analogy to the NRPS enzyme systems, it is possible for example for the novel polyketide synthases prepared according to the invention to be employed for synthesizing an abundance of polyketide compounds such as, for example, macrolides, and for the hybrid NRPS-PKS enzyme systems to be employed for synthesizing peptide-polyketide mixed structures. In addition, enzyme-catalyzed reactions on particular natural substances are also conceivable, with modifications such as, for example, hydroxylations, transaminations, inter alia, taking place on these substrates through use of polypeptides obtained according to the invention.

The invention additionally relates to the use of the product compound obtained in this way for testing the pharmacological activity thereof. Thus, for example, novel PKS enzyme systems prepared by the method of the invention can be employed for synthesizing novel macrolide compounds. The macrolides obtained in this way can then be tested for example for their antibiotic activity. It is clear to the skilled worker that it is particularly advantageous for this purpose if the number of different product compounds such as, for example, macrolides available for these tests is as large as possible. This can be achieved most advantageously by the combinatorial approach described previously, initially preparing a large number of DNA which code for enzymes of a particular class, such as, for example, PKS enzyme systems, obtaining a large number of polypeptides, such as, for example, PKS enzyme systems, by expression of these DNA, using these polypeptides to synthesize product compounds such as, for example, macrolides, investigating these product compounds, e.g. by a high throughput test method, for pharmacological activity, and thus obtaining, for example, novel macrolide antibiotics.

The present invention is to be illustrated and described in detail below by means of exemplary embodiments. The method of the invention for preparing a DNA which codes for a polypeptide composed of a plurality of sections in a circular DNA vector can be employed particularly advantageously for preparing DNA which codes for modular enzyme systems. The following exemplary embodiments show the preparation of DNA coding for non-ribosomal peptide synthetases, with the linkage according to the invention of the DNA fragments taking place in a bacterial plasmid sector.

EXPLANATIONS OF THE SEQUENCE LISTINGS AND THE FIGURES

FIG. 1 shows typical NRPS and PKS (type 1) modules and the arrangement of their domains.

FIG. 2 shows two typical representatives of NRPS and PKS (type 1) systems.

FIG. 3 shows the construction of a module library for the MCA method.

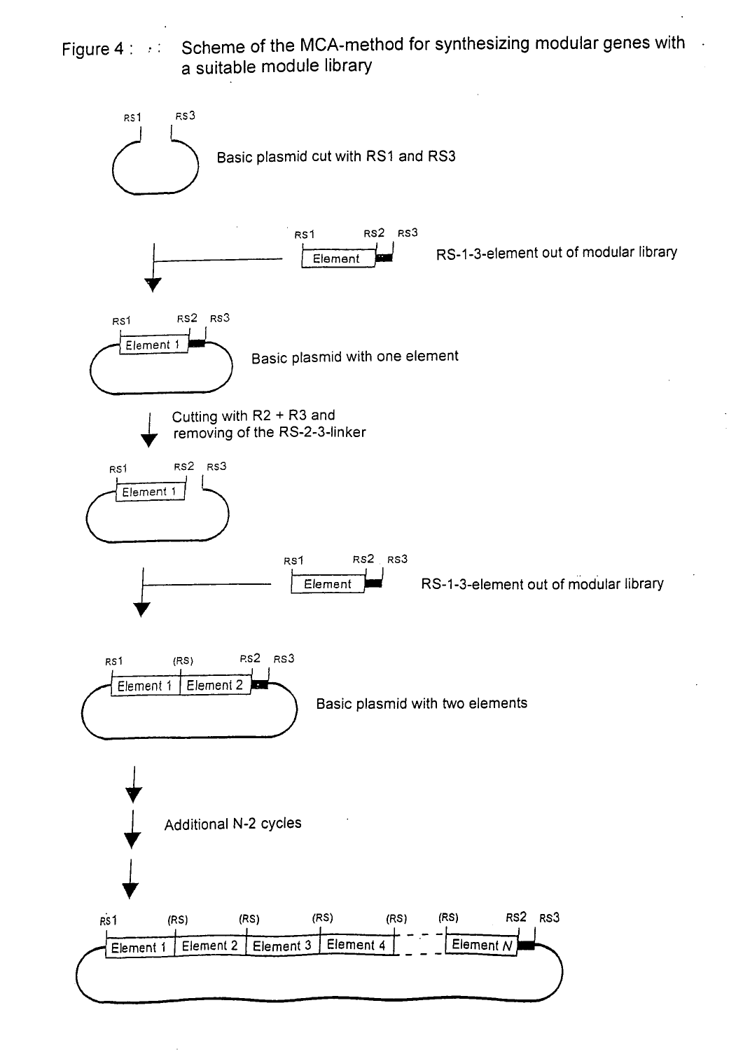

FIG. 4 shows the scheme of the MCA method for synthezising modular genes with a suitable module library.

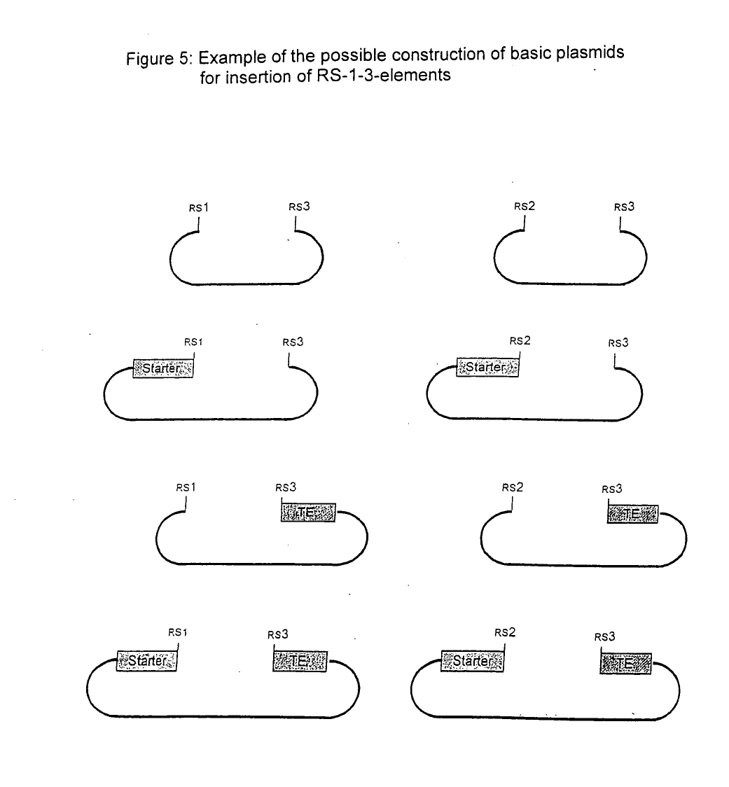

FIG. 5 shows an example of the possible construction of basic plasmids for insertion of RS-1-3 elements.

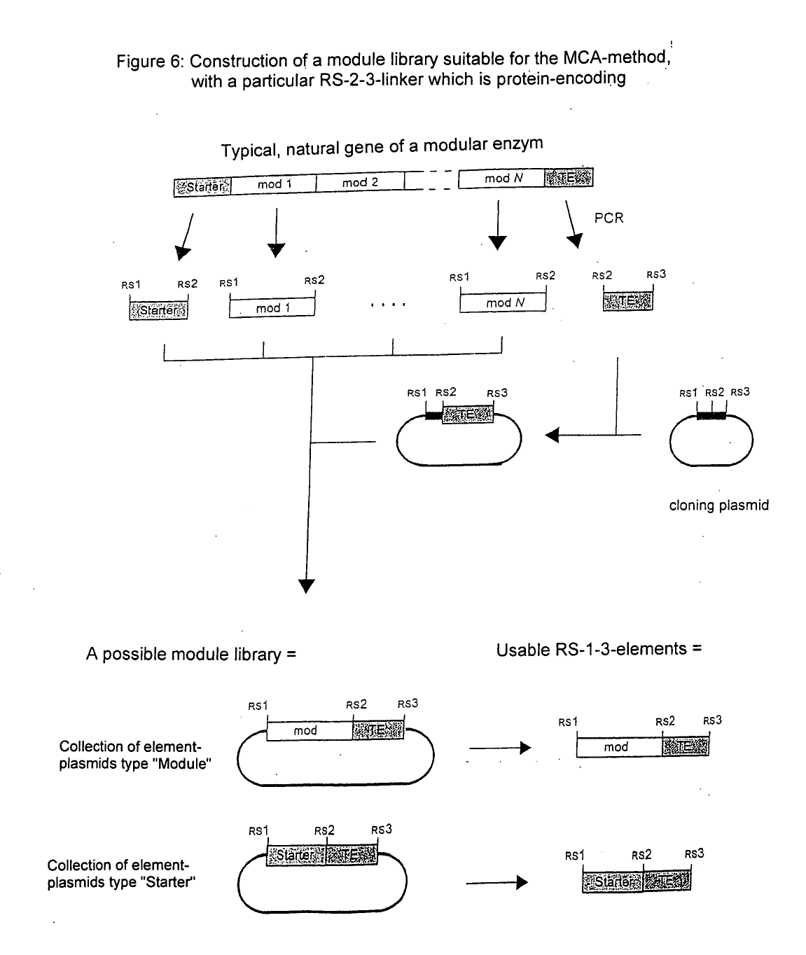

FIG. 6 shows the construction of a module library suitable for the MCA method, with a particular RS-2-3 linker which is protein-encoding.

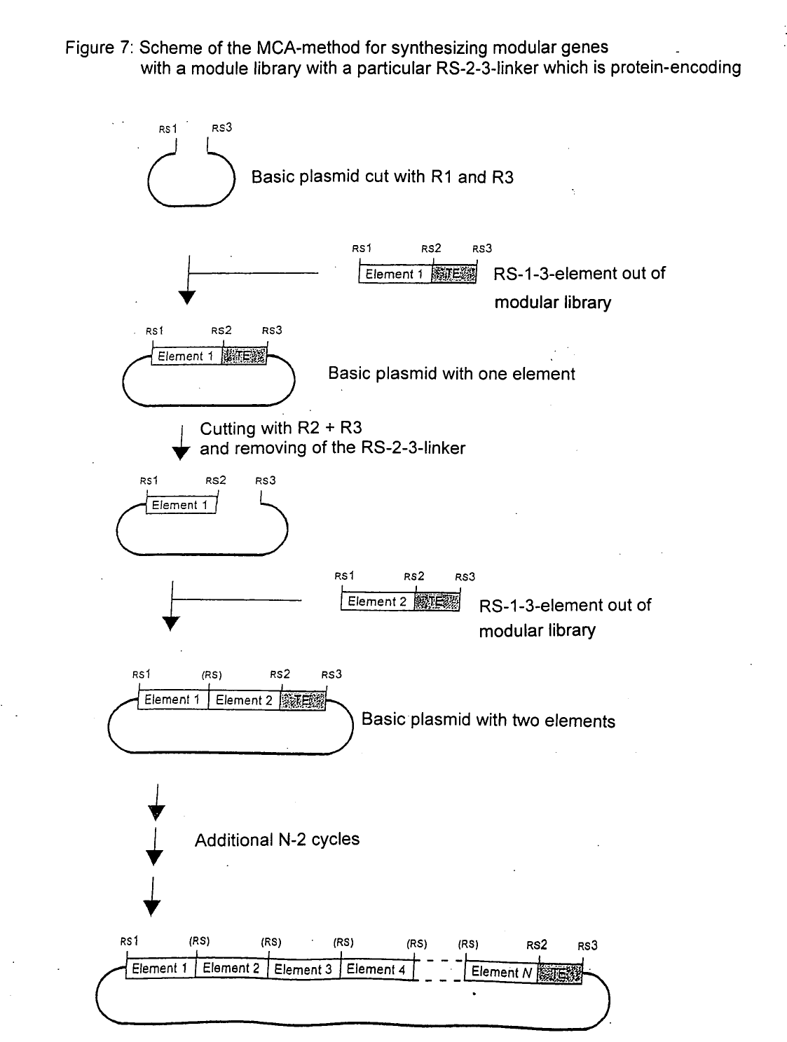

FIG. 7 shows the scheme of the MCA method for synthesizing modular genes with a module library with a particular RS-2-3 linker which is protein-encoding.

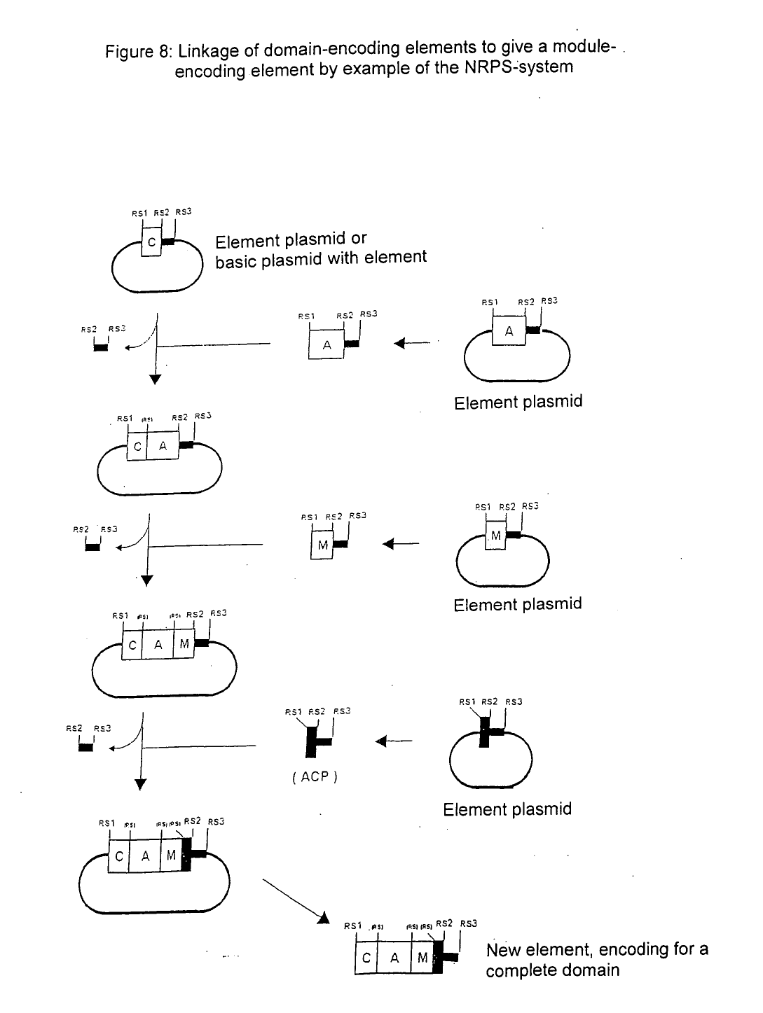

FIG. 8 shows the linkage of domain-encoding elements to give a module encoding element by example of the NRPS system.

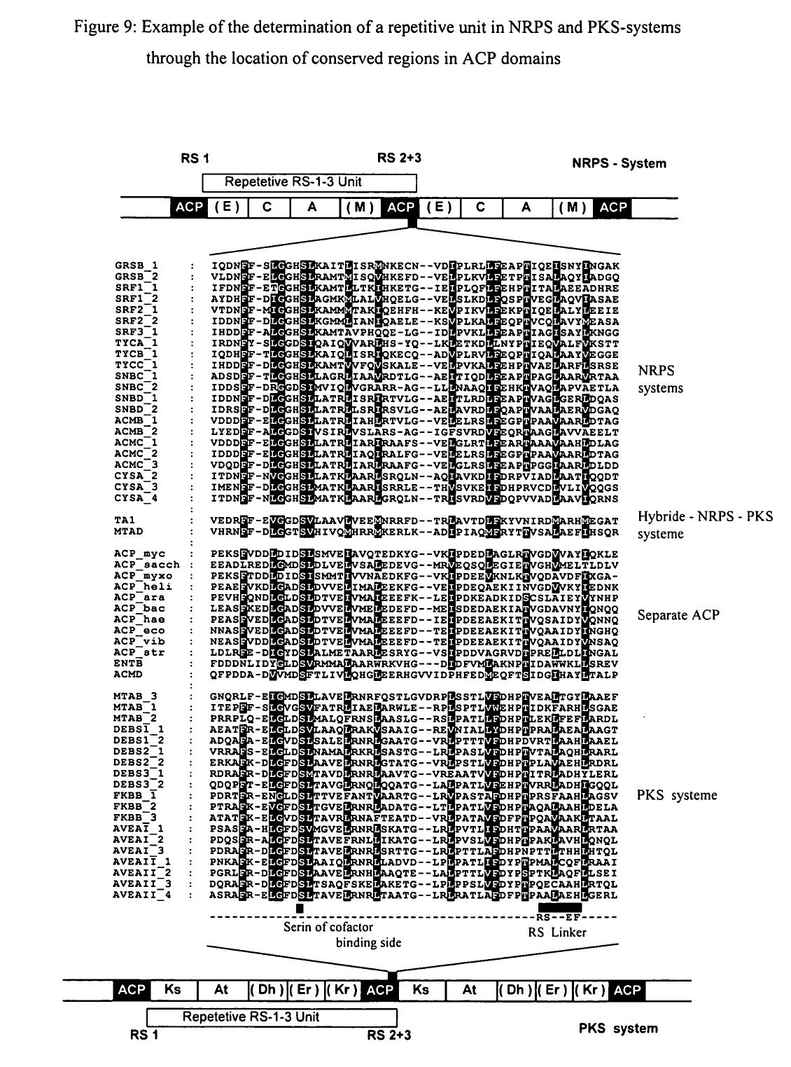

FIG. 9 shows an example of the determination of a repetitive unit in NRPS and PKS systems through the location of conserved regions in ACP domains.

The depicted protein sequences are derived from the proteins listed below, and the numerical index following the protein name in the sequence comparison refers to the number of the module within the particular PKS or NRPS. Amino acid positions at least 70% occupied by similar amino acids have a black background. The database numbers of the sequences in the public databases “GenBank” or “SwissProt” are indicated in parentheses, and ACP proteins from fatty acid biosynthesis are indicated by a following FA:

| | | |
|---|---|---|
| GRSB | Gramidicin S synthetase II | P14688 |
| | from Bacillus brevis | |
| SRF 1, 2, 3 | Surfactin synthetase 1, 2, 3 | P27206, |
| | from Bacillus subtilis | Q04747, |
| | | Q08787, |
| ACM B, C | Actinomycin synthetase II, III | AF047717, |
| | from Streptomyces chrysomallus | AF204401 |
| CYS A | Cyclosporin synthetase | Z28383 |
| | from Tolypocladium niveum | |
| TA1 | TA1 protein (NRPS + PKS) | Q9Z5F4 |
| | from Myxococcus xanthus | |
| MTAD | MTAD protein (NRPS + PKS) of | AF188287 |
| | myxothiazol biosynthesis | |
| | from Stigmatella aurantiaca | |
| ACP_myc | Acyl carrier protein | Q10500 (FA) |
| | from Myobacterium tuberculosis | |
| ACP_sacch | Acyl carrier protein | P11830 (FA) |
| | from Saccharopolyspora erythrea | |
| ACP_myxo | Acyl carrier protein | P80921 (FA) |
| | from Myxococcus xanthus | |
| ACP_heli | Acyl carrier protein | P56464 (FA) |
| | from Helicobacter pylori | |
| ACP_ara | Acyl carrier protein | P53665 (FA) |
| | from Arabidopsis thaliana | |
| ACP_bac | Acyl carrier protein | P80643 (FA) |
| | from Bacillus subtilis | |
| ACP_hae | Acyl carrier protein | P43709 (FA) |
| | from Haemophilus influenzae | |
| ACP_eco | Acyl carrier protein | P02901 (FA) |
| | from Escherichia coli | |
| ACP_vib | Acyl carrier protein | P55337 (FA) |
| | from Vibrio harveyi | |
| ACP_str | Acyl carrier protein | Q02054 |
| | from Streptomyces coelicolor, | |
| | actinorhodin biosynthesis | |
| ENTB | Acyl carrier protein | P15048 |
| | from E. coli, section of the | |
| | bifunctional isochorismatase in | |
| | enterobactin biosynthesis | |
| ACMD | Acyl carrier protein | AF134588 |
| | from Streptomyces chrysomallus, | |
| | actinomycin biosynthesis | |
| MTAB | Polyketidesynthase of | AF188287 |
| | myxothiazol biosynthesis | |
| | from Stigmatella aurantiaca | |
| DEBS 1, 2, 3 | Polyketide synthases 1, 2, 3 of | M63676, |
| | 6-deoxyerythronolide B | M63677 |
| | biosynthesis | |
| | from Saccharopolyspora erythrea | |
| FKBB | FK506 polyketide synthase | AF082100 |
| | from Streptomyces sp. MA6548 | |
| AVAE I, II | Avermectin polyketide synthases | AB032367 |
| | from Streptomyces avermitilis | |

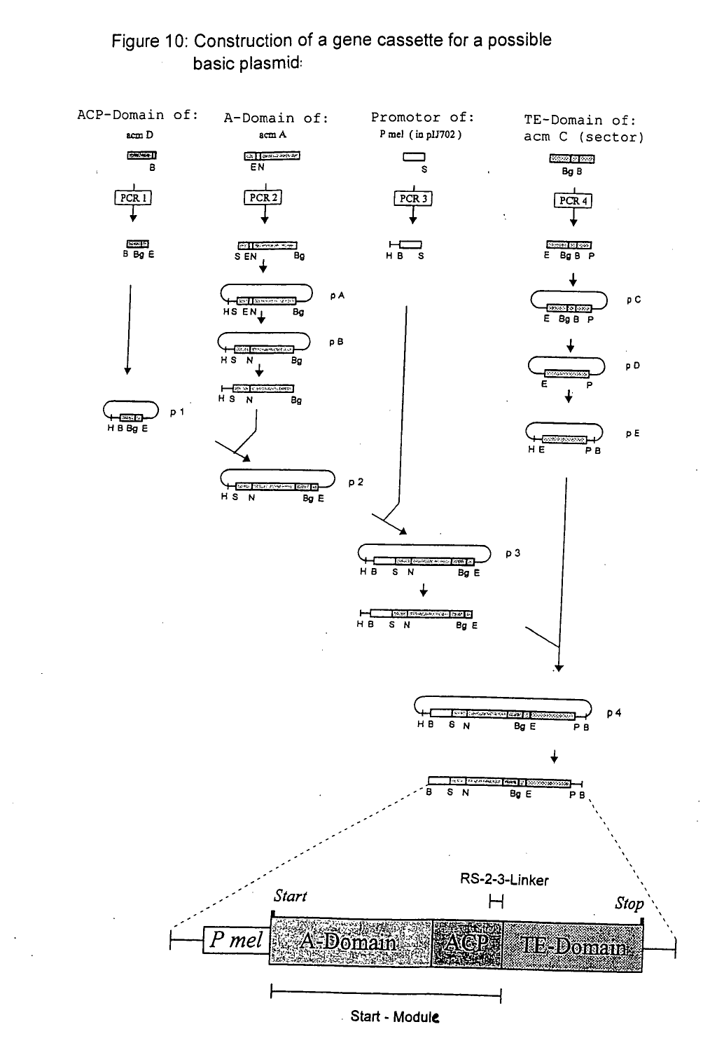

FIG. 10 shows the construction of a gene cassette for a possible basic plasmid

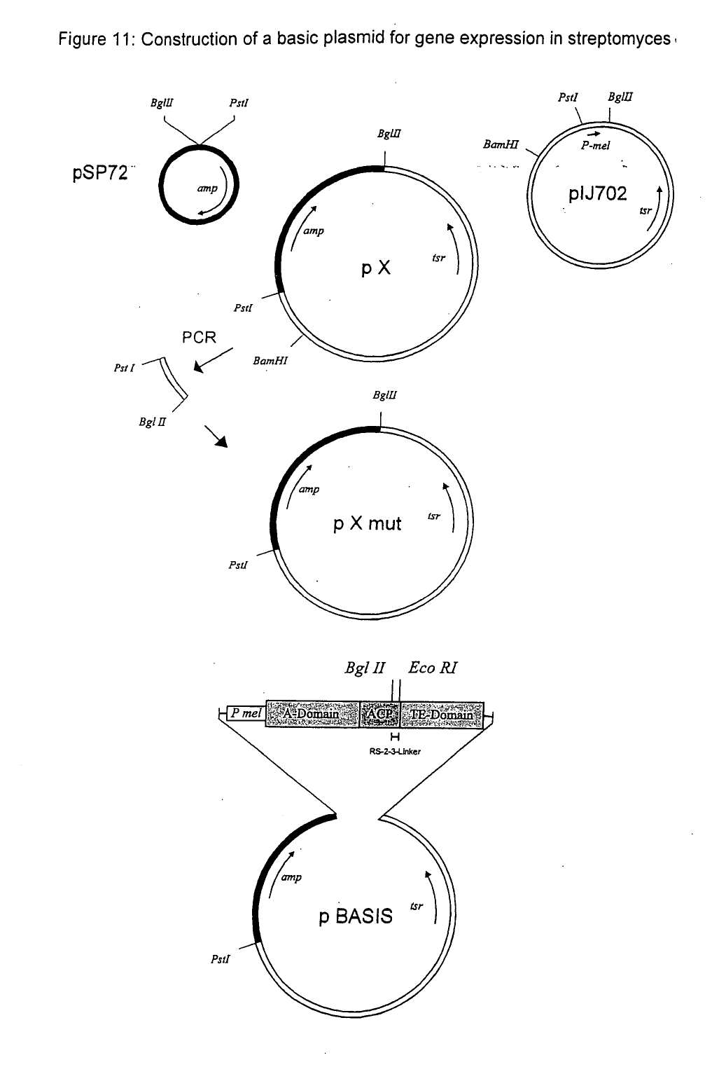

FIG. 11 shows the construction of a basic plasmid for gene expression in streptomyces.

The method of the invention referred to hereinafter as module cycle addition method (MCA) allows enzyme systems with a modular structure, such as, for example, non-ribosomal peptide synthetases, to be prepared with distinctly less effort than in methods known in the prior art. The MCA method has the great advantage in particular of in vitro fusion of modules, domains or other part-regions at defined points. The novel gene which is assembled in vitro therefore codes for a fixed module arrangement in the enzyme and can then be used in vivo for synthesizing novel enzymes.

It is advantageous for the MCA method if it is possible to start from a collection which is as comprehensive as possible of DNA fragments which code for individual modules, domains or only of parts of domains of enzymes systems with a modular structure. Such a collection of defined DNA fragments is referred to hereinafter as a module library.

Such DNA fragments can be generated by conventional methods in molecular biology, such as, for example, by means of the PCR technique [13-17], and also modified at the same time. Each DNA fragment of the module library can be cloned, for replication, storage, modification and recovery, in a conventional cloning plasmid such as, for example, in pUC plasmids for E. coli. For this purpose, the sequence of the DNA fragment is modified at both ends in such a way that it can be integrated, by using defined restriction cleavage sites, into the cloning site of cloning plasmids, and can be cut out again as required. The use of restriction enzymes and the modifications of DNA sequences are standard methods of molecular biology and are also used—in example 1-4. A plasmid in which a complete element is present for use in the MCA method is referred to hereinafter as an element plasmid (concerning this, see also FIGS. 3 and 4).

An element plasmid used in the MCA method has three different restriction cleavage sites (RS1, RS2, RS3), each of which is recognized and cleaved by a different restriction enzyme (R1, R2, R3). The cleavage site RS1 forms the start of the element, and RS2 and RS3 form, in this sequence, the end of the element, it being possible for RS3 also to be located in the plasmid. The region between RS2 and RS3 is referred to hereinafter as RS-2-3 linker. It is possible in this way to isolate the element for example as RS1-RS2 DNA fragment (RS-1-2 element) or, together with the RS-2-3 linker, as RS1-RS3 DNA fragment (RS-1-3 element). Elements which additionally comprise RS1, RS2 or RS3 cleavage sites must have these additional cleavage sites deleted beforehand by targeted mutagenesis. Methods of targeted DNA mutagenesis form part of the conventional techniques of molecular biology [16-20]. The choice of the restriction enzymes R1, R2 and R3 must take place according to the following three criteria:

(i) The DNA ends generated by the restriction enzymes R1 and R2 must be compatible, i.e. they can be linked together by use of a DNA ligase in vitro.

(ii) The linking sequence produced by linkage of RS1 and RS2 should not in this case be cleavable by any of the three restriction enzymes used (R1, R2, R3).

(iii) It must be ensured that the DNA ends generated by R2 and R3 are not compatible.

The three restriction enzymes can otherwise be chosen as desired (e.g. R1=BamHI; R2=BglIII; R3=EcoRI), but they must be chosen uniformly for all elements of a module library. This results in all elements of a module library having the property of being linkable to multimers by the MCA method, and the resulting multimer can in turn be used as independent novel element in the MCA method. All linked elements within the multimer have the same orientation because the MCA method always links the end of one element to the start of the subsequent element. These linkages take place in plasmids suitable for this purpose, which are referred to hereinafter as basic plasmids (concerning this, see also FIG. 5).

The simplest case of a basic plasmid has only the two restriction cleavage sites RS1 and RS3. The region between RS1 and RS3 can be deleted and replaced by an RS-1-3 element which is derived from an element plasmid of the module library (FIG. 4). This results in a basic plasmid which comprises a single element. It is analogously possible for a basic plasmid also to have only the two restriction cleavage sites RS2 and RS3 (FIG. 5), and for the intervening region to be deleted and replaced by an RS-1-3 element, because the RS1 and RS2 cleavage sites are compatible. For every further element to be inserted in one of these basic plasmids it is necessary in principle to carry out the following three steps:

(i) cutting with the restriction enzymes R2 and R3;

(ii) deletion of the RS-2-3 linkers; and

(iii) insertion of an RS-1-3 element.

Performance of these three steps is referred to hereinafter as cloning cycle. The procedure for a cloning cycle can easily be carried out with conventional methods of molecular biology and is demonstrated in example 4.

A DNA sequence coding for a protein or a protein section is generally referred to as DNA reading frame. Each element from the module library has a reading frame with RS1 located at its start and RS2 located at its end. It is therefore necessary for RS1 and RS2 in the elements to be matched with one another so that a continuous reading frame is produced again after a linkage of RS2 ends with RS1 ends as described above. Each cloning cycle thus leads to a corresponding extension of the reading frame, which then codes for a single protein comprising all the linked elements. In order that the reading frame generated in the basic plasmid also leads to the synthesis of an appropriate protein it is necessary for the reading frame to be expressed. It is possible to use for this purpose a basic plasmid which already has a DNA control region (promoter) necessary for expression. The start of the reading frame (start codon) and the end of the reading frame (termination codon) may likewise already be part of a basic plasmid used. In this case, none of the elements used in the MCA method needs to have a start codon or termination codon. It is likewise possible for the start of the reading frame also to be located in the element which is inserted first into the basic plasmid. Such an element which is inserted first may be, for example, a starter module. The end of the reading frame may also be located inside an inserted element which, for example, is inserted last into the basic plasmid. Suitable for this in the NRPS enzyme systems is, in particular, a terminating thioesterase (TE) domain or, where appropriate, an amide synthase. This also applies moreover to the polyketide synthases which likewise have a modular structure. Basic plasmids can also be designed so that, even before integration of the first element, they already comprise fixed reading frames for the start and end of a protein. Such integral start and end regions of the basic plasmids may be, for example, a starter module at the start and a TE domain at the end (FIG. 5), and integration of elements from the module library then takes place between the start and end regions.

It is common to all basic plasmids which have the termination codon for the reading frame generated by the MCA method behind the RS3 cleavage site that the RS-2-3 linker of the element inserted last also becomes part of the generated reading frame (see also FIG. 4). It is therefore necessary for the RS-2-3 linker sequence likewise to be protein-encoding and for the location of the RS2 and RS3 cleavage sites to be matched to one another so that, after integration of the last element, the reading frame continues beyond this region and up to the termination codon present on the basic plasmid. If such basic plasmids are to be used in the MCA method, this must be taken into account in the construction of the module library, and the length of the RS-2-3 linker must be chosen as short as possible. However, if the termination codon is located inside the element integrated last, the RS-2-3 linker sequence does not play a crucial part because it is then no longer part of the reading frame formed.

For optimal integration of the linker sequence into the resulting reading frame, it is, however, also possible to choose the linker sequence such that the linker itself codes for a domain, a module or for part-regions thereof. In the cases of the NRPS and PKS systems, the RS-2-3 linker may code, for example, for a complete TE domain (FIG. 6). Since each element of the module library has this linker, every reading frame generated by the MCA method will thus automatically be terminated by a sequence coding for a TE domain (FIG. 7). The end of the reading frame may in this case also simultaneously be located inside the RS-2-3 linker, that is to say, for example, at the end of the sequence coding for the TE domain and before the RS3 cleavage site. It is also possible in principle to use every other part-region of the repetitive sections of modular systems themselves as RS-2-3 linkers. As the simplest example, the region coding for the ACP domains, or parts thereof, for example, can serve as linker sequence, as described in example 1-4.

The described requirements concerning the RS-2-3 linker thus imply a uniform structure of the elements within a module library. The use of the RS2 and RS3 cleavage sites makes it possible, however, for an already existing module library to be provided with other linkers in a simple way. This makes it possible for existing module libraries to be adapted simply for use with novel basic plasmids which are available, for example, only after completion of a module library.

The properties of the RS1, RS2 and RS3 cleavage sites described above mean that all the elements of a module library which are linked by the MCA method subsequently behave like a new RS-1-3 element which can be used freely within the module library for combining with other elements. The linkage of elements therefore need not necessarily take place in basic plasmids, but can be carried out even with element plasmids. Elements coding for whole modules can be assembled for example to multimodular elements and then resubmitted, as multimodular block, to the MCA method later. This makes it easily possible to define as multimodular block a core region, which is regarded as important, of an enzyme, and to prepare numerous enzyme derivatives with this constant core region by the MCA method.

It is also possible in principle for a module library to be designed so that its original elements code not for complete modules but only for domains or part-regions thereof (FIG. 8). Linkage of these original elements then allows in principle the assembly of DNA fragments which code for all conceivable domains or modules; For example, all known PPS modules can in this way be equipped with an additional activity for N-methylation or epimerization of substrates. It is thus possible for a module library also to be supplemented by novel elements without the need for other DNA sources foreign to the module library for this purpose.

Every module library which meets the criteria described above can be used with appropriately matched basic plasmids for generating novel enzymes. This entails another element being introduced into the basic plasmid with each MCA cloning cycle. The efficiency of the MCA method can be increased by using basic plasmids which permit the cloning cycles to be carried out in vivo in organisms which are particularly suitable for this purpose. An organism which is particularly suitable and a host which is established as standard in molecular biology is, for example, Escherichia coli. Numerous selection markers and autonomously replicating plasmids which can be used for producing basic plasmids are available for this organism. Cloning in this host, comprising transformation, cultivation and DNA isolation, is possible with simple technical means and requires only simple basic knowledge of molecular biology. Expression of the enzyme genes generated by the MCA method can in turn take place in other organisms which are particularly suitable for synthesizing natural substances. Organisms of this type are, for example, streptomyces, for which numerous autonomously replicating plasmids, selection markers and promoter elements are likewise available. Both advantages can be utilized simultaneously in the MCA method, by constructing basic plasmids which replicate in both organisms used and harbor a promoter region which is suitable, for example, for Streptomyces. Plasmids which replicate in at least two different organisms and can be exchanged between these organisms are generally referred to as shuttle plasmids. Numerous shuttle plasmids have already been described for E. coli and streptomyces [5, 21-22], some of which also harbor promoter elements for streptomyces. An appropriate construction of basic plasmids which are suitable for the MCA method can therefore take place on the basis of shuttle plasmids which are already available. However, it is also possible in principle for any E. coli plasmid and any streptomyces plasmid to be combined to give a novel shuttle plasmid, as described in example 1.4.

A single basic plasmid which, after insertion of a novel element, immediately has a reading frame suitable for expression can be used after each cloning cycle directly for expression or for further extension in the MCA method. Transformation into a suitable organism such as, for example, E. coli makes in vivo replication of the individual basic plasmid easily possible. It is possible in this way to obtain a sufficient amount of basic plasmid for subsequent extension in the MCA method and to allow in parallel in vivo expression of the novel gene. Gene expression can take place for example by transformation into streptomyces if a basic plasmid with the property of a shuttle plasmid is used. However, in principle, in vivo replication and gene expression can also be carried out in a single organism if the basic plasmid used replicates in this organism and has the necessary promoter elements.

Because of the properties of the RS-1-3 elements, insertion of the novel element into the basic plasmid used takes place in the correct orientation in relation to the reading frame. The plasmids obtained after a cloning cycle therefore need no further examination but can be used immediately for a subsequent MCA cloning cycle. Because of the properties of the RS1, RS2 and RS3 cleavage sites, however, unwanted simultaneous insertion of 3+2 n elements (n=0 to infinity) is also theoretically possible in a cloning cycle. Unwanted insertions of this type can be substantially avoided through the choice of suitable ligation conditions, however [17]. A further possibility in general is to dephosphorylate the elements used in the MCA method, before insertion for ligation with basic plasmids, using a method customary in molecular biology. This. prevents the formation of multiple insertions.

Since the MCA method does not require elaborate checks of the basic plasmid obtained after a cloning cycle, it is possible for cloning cycles to be carried out standardized-and in parallel. This property of the MCA method promotes automation of the MCA method. Because of the simple standardization, the cloning cycles can also be carried in the form of a purely random approach. For this purpose it is possible, for example, to mix an amount of identical basic plasmids for in vitro ligation with an appropriate amount of different RS-1-3 elements. It is also possible likewise to mix a mixture of different basic plasmids for in vitro ligation with one sort of identical or different. RS-1-3 elements. After the in vitro ligation, the mixture can be transformed directly into a suitable organism, for example for replication in E. coli or for gene expression in streptomyces. Every transformant obtained normally harbors only one sort of plasmid. Isolation of the plasmids from the individual transformants allows the different basic plasmids to be separated again. Alternatively, the plasmids can also be isolated from a mixture of transformants, again resulting in a mixture of basic plasmids. This mixture of basic plasmids can in turn be employed directly in a random MCA cloning cycle.

Exemplary Embodiments 1-4:

The NRPS genes of actinomycin biosynthesis which were used in the exemplary embodiments are derived from chromosomal DNA of the strain Streptomyces chrysomallus. This strain is deposited in the American Type Culture Collection under the ATCC number 11523. The sequences of the NRPS genes used are deposited in the GenBank database under the database entries AF134587 (acMA), AF047717 (acmB), AF204401 (acmC) and AF134588 (acmD). Chromosomal S. chrysomallus DNA cloned in the cosmids pA1, pP1 and subclones derived therefrom is used for the PCR [4]. The cloning steps necessary for assembling the gene segments take place in the E. coli strain DH5a (GibcoBRL). Expression of the NRPS genes takes place in Streptomyces lividans TK64 (John Innes Collection). The cloning plasmids used are pTZ18 (Pharmacia), pSP72 (Promega, Mannheim), pBluescript SK+ (Stragagene), pSL1180 (Pharmacia) and pIJ702 [24]. All the methods of molecular biology used are standard methods [17, 25] and are to be found in appropriate textbooks.

### Example 1

### Preparation of a Basic Plasmid Which Already Codes for a Start Module and a TE Domain for Gene Expression in Streptomyces.

The synthesis of a basic plasmid (pBASIS) which has the properties of a shuttle plasmid and makes it possible to use the restriction cleavage sites RS1=BamH1, RS2=BglII and RS3=EcoRI in the MCA method is described below for the MCA method. The basic plasmid is intended to code simultaneously for a starter module and a TE domain located at the end. Between these two regions it is intended to be able to insert RS-1-3 elements from NRPS systems, specifically into an RS-2-3 linker which is already present on the basic plasmid and which is protein-encoding.

The basic plasmid is intended to be usable immediately after each cloning cycle for gene expression in streptomyces, but the possibility of recovery of individual inserted RS-1-3 elements as assembled RS-1-3 element is not provided for.

The plasmid portion necessary for replication in E. coli is derived from pSP72 (Promega) and makes ampicillin selection possible. The portion necessary for replication in streptomyces, and the streptomyces promoter (P-mel) are derived from pIJ702 [24] and make thiostrepton selection and expression of the gene thus generated by the MCA method possible. The fixed point used to determine the junctions (position of the RS-2-3 linker sequence) between the repetitive units of a modular NRPS system-in this example is the-C-terminal region of ACP domains. This region is not only conserved for the ACP domains within an NRPS system but also shows great homologies with PKS systems and separately present ACP domains. Comparison of the gene-encoded protein sequences (alignment) allows the strictly conserved serine (S), necessary for binding of the cofactor 4′-phosphopantethein, to be determined exactly (FIG. 9). The RS-2-3 linker sequence is chosen in this example so that it codes for 6 amino acids, of which the first two (arginine=R and serine=S) are encoded by the RS2 cleavage site (BglII) and the last two (glutamate=E and phenylalanine=F) are encoded by the RS3 cleavage site (EcoRI) The region encoded by the RS-2-3 linker is located in the individual ACP domains between 29 and 32 amino acids behind the strictly conserved serine of the cofactor binding site, and the position of the RS2 and RS3 cleavage site can be determined for each ACP domain by an alignment, shown in FIG. 9. The position of the chosen RS1 cleavage site (BamHI) is identical to the position of RS2 and likewise codes for arginine and serine. It is possible in this way by a simple alignment to determine the location of the RS1, RS2 and RS3 cleavage sites, which must be introduced by mutagenesis into gene fragments in order for the latter to be useful as RS-1-3 elements for the basic plasmid constructed in this example. This basic plasmid is constructed in several stages, which are described in more detail below.

1.1 Construction of a DNA Fragment Which Codes for a Start Module Composed of Individual Domains

The initiation module of actinomycin biosynthesis, which is present in the natural synthesis system in the form of two separate protein components, ACMS I and AcmACP, is to be used as starter module of the basic plasmid (see also FIG. 2). Together, the two proteins afford an activation domain which activates and covalently links 4-hydroxy-3-methylanthranilic acid. This activation domain is to be prepared by fusion of the appropriate genes. For this purpose, firstly the AcmACP gene (acmD) is amplified by PCR using the DNA oligomers (primers) 5′-GGCGGATCCATCTCGAAGGACGACATGAG-3′ and 5′-AGGAATTCGTGGATAGATCTGATCGAGGTGA-3′ (PCR 1 in FIG. 10). This PCR converts the start of the acmD gene into a BamHI cleavage site, and the internal BamHI cleavage site at position 197 is mutagenized. The introduced BamHI cleavage site is used for the fusion with the ACMS I gene (acmA) which is described later. The PCR simultaneously introduces a BglII cleavage site at position 193 and an EcoRI cleavage site at position 205. The location of this BglII and EcoRI cleavage site introduced into the acmD gene has been determined by the previously described comparison of the AcmACP sequence with ACP consensus sequences (see also FIG. 9) and form the RS-2-3 linker of the latter basic plasmid. The ACP domain which has been truncated thereby is restored by the later insertion of appropriate RS-1-3 elements.

```
acmD sequence in S. chrysomallus:
bp 1 10 197
            : : :
      CTCGTGATCTCGAAG......TTCACCTCGATCGACGGGATCCACGCCTACCTCACGGCGCTG...
            M I S K F T S I D G I H A Y L T A L
                                                                                BamHI
acmD sequence in plasmid p1:
bp 1 10 193 205
            : : : :
      GGATCCATCTCGAAG......TTCACCTCGATCAGATCTATCCACGAATTC
            S I S K F T S I R S I H E F
          BamHI Bg1II EcoRI
                                                                            RS-2-3 linker region
```

The numbering of the base pairs (bp) relates to the original GTG start codon of the acmD gene, and the encoded amino acid sequence is indicated underneath the DNA sequence. The resulting PCR fragment is cut with BamHI+EcoRI and cloned into pTZ18 (BamHI+EcoRI), resulting in plasmid p1.

The ACMS I gene. (acmA) is amplified by PCR using the primers 0.5°-AGGAAGCTGGCATGCCCGATAAATGGT-3′ and 5′-ATGTCGTCCTTCGAGAAGATCTGGCC-3′ (PCR 2 in FIG. 10). This converts the start of the gene into an SphI cleavage site, and a BglII cleavage site is introduced at position 1414. The SphI cleavage site is used for the fusion with the promoter region, which is described later, and the introduced BglII cleavage site is used for the fusion with the mutagenized acmD gene, which is described later:

```
acmA sequence in S. chrysomallus:
bp 1 10 265 291 1399 1414
                  : : : : : :
      GGTATGGCCGATAAA.......GAATTC....GCGGCCGC....GAGCTCAAGGGGGCCTCGTGA
            M A D K E F R P E L K G A S *
                                                    EcoRi NotI
acmA sequence in PCR fragment:
bp 1 10 265 291 1399 1414
              : : : : : :
        GCATGCCCGATAAA.......GAATTC....GCGGCCGC....GAGCTCAAGGGGGCCAGATCT
            M P D K E F R P E L K G A R B
            SphI EcoRI NotI BglII
```

The numbering of the base pairs (bp) relates to the original ATG start codon of the acmA gene, and the encoded amino acid sequence is indicated underneath the DNA sequence. The TGA termination codon is identified by an Asterix. The resulting PCR fragment is cut with SphI+BglII and cloned into pSP72 (SphI+BglII), resulting in plasmid pA. To delete an internal EcoRI cleavage site, the region from the SphI cleavage site up to the NotI cleavage site from plasmid pA is amplified by PCR using the primers 5′-AGCTGAAGCTTGCATGCCCGATAAATGGTGG-3′ and 5′-ACCAGGTACTGCCGGCCGCACACGCTCCACCAGAGGCTCGAACTCG-3′. The sequence of the EcoRI cleavage site in the PCR product is changed by the primers from 5′-GAATTC-3′ to 5′-GAGTTC-3′. The PCR product is cut with a SphI+NotI, and the corresponding region in plasmid pA is replaced by the cut PCR product. The result is plasmid pB:

```
acmA sequence in plasmid pB:
bp  1 10 265 291 1399 1414
              : : : : : :
          GCATGCCCGATAAA.......GAGTTC....GCGGCCGC....GAGCTCAAGGGGGCCAGATCT
              M P D K E F R P E L K G A R S
            SphI NotI BglII
```

The acmA gene mutagenized in this way is cut as HindIII-BglII fragment out of plasmid pB and cloned into plasmid p1 (HindII+BamHI). This results in plasmid p2. Fusion of the BglII and BamHI cleavage sites results in a sequence which can no longer be cut with these enzymes. The fusion gene between acmA and acmD present in plasmid p2 thus codes for an almost complete module, i.e. the encoded enzyme contains the complete adenylation domain of ACMS I and the ACP domain of AcmACP at the end of the RS-2-3 linker.

1.2 Attachment of a Promoter Element

The promoter of the melanin operon (P-mel) from plasmid pIJ702 [24], which has already been used for the expression of NRPS genes [4, 26], is to be used as promoter for gene expression in streptomyces. The promoter region is amplified from pIJ702 by PCR using the primers 5′-GCCAAGCTTCCGGGATCCGCTCGCCCGGCCGCCGGTCCCCCTG-3′ and 5′-TTCCGGCATGCGGGACCTCCTGGGTGC-3′ (PCR 3 in FIG. 10). This PCR introduces a HindIII and a BamHI cleavage site in the 5′ region of the promoter, and a natural SphI cleavage site in the 3′ region is retained. This SphI cleavage site is used for the fusion of the promoter region with the mutagenized start of the acmA gene in plasmid p2, which is described below:

```
mel-P Promoter region in PCR fragment:
bp 1 407
        : :
        AAGCTTCCGGGATCCGCTCGCCCGG..........AGGAGGTCCCGCATGC
        HindIII BamHI SphI
```

The PCR product is cut with HindIII+SphI and cloned into plasmid p2 (HindIII+SphI). This results in plasmid p3.

1.3 Attachment of an Element Encoding TE Domains

The DNA fragment which codes for the TE domain is to be obtained from the ACMS III gene (acmC). The TE domain is located in the C-terminal end of the enzyme (see also FIG. 2), and the junction between the repetitive unit and the terminally located TE domain is fixed by the ACP domain of the last repetitive element in the enzyme, that is to say the MeVal module (mod 5). Accordingly, only the terminal region of the acmC gene is amplified by PCR, and thereby an RS3 (EcoRI) cleavage site which corresponds to the location of RS3 in the RS-2-3 linker (bp position 11908 in the acmC gene) is introduced. A second restriction cleavage site (PstI) introduced by the PCR is to be located behind the termination codon of the acmC gene and is used only for further cloning. The PCR (PCR 4 in FIG. 10) is carried out with a primer combinataion 5′-GCCGAATTCCTGGACCTCGACGACCCGGA-3′ and 5′-CTTCTGCAGGTCGACGGAGAGCACATCGGT-3′:

```
acmC sequence in S. chrysomallus:
bp 11896 11908 12488 12701 12742 12843
                        : : : : : :
            ACGCCGGGCGGGATCGCCGCCCGGCTGGAC...GAGATCT...CGGATCC...TGA...CTGGAG
            T P G G I A A R L D E I R I *
                        ------------------ BglII BamHI
                          (RS-2-3 linker region)
acmC sequence in PCR fragment:
bp 11908 12488 12701 12742 12543
                                : : : : :
                                GAATTCCTGGAC...GAGATCT...CGGATCC...TGA...CTGCAG
                                E F L D E I R I *
                                Ecori BglII BamHI PstI
```

The numbering of the base pairs (bp) relates to the original ATG start codon of the acmC gene, and the encoded amino acid sequence is indicated underneath the DNA sequence. The TGA termination codon is identified by an Asterix. The PCR product is cut with EcoRI+PSTI and cloned for further mutagenesis into plasmid pALTER1 (EcoRI+PStI), resulting in plasmid pC; the plasmid pALTER is part of a mutagenesis system which is described in detail in example 2. The use of this mutagenesis system mutagenizes the two natural cleavage sites BglII and BamHI which are present in the PCR product without altering the amino acid sequence encoded by the EcoRI-PstI fragment, resulting in plasmid pD.

```
acmC sequence in plasmid pD:
bp 1908 12488 12701 12742 12843
                                : : : : :
                                GAATTCCTGGAC...GAAATCT...CGCATCC...TGA...CTGCAG
                                E F L D E I R I *
                                EcoRI PstI
```

The EcoRI-PstI fragment mutagenized in this way is recloned from plasmid pD into pBluescriptSK+ (EcoRI+PstI), resulting in plasmid pE. The plasmid pE thus codes for the C-terminally located TE domain of ACMS III and the remainder of the ACP domain, located in front, of the MeVal module, starting with the RS3 position (EcoRI) of the RS-2-3 linker region. The plasmid pE is cut with HindIII+EcoRI, and the HindIII-EcoRI fragment from plasmid p3 is inserted. This results in plasmid p4 which codes for complete NRPS domains which are covalently connected together in the sequence activation domain, ACP domain and TE domain (FIG. 10). The RS-2-3 linker region is defined by the BglII and EcoRI cleavage sites, which are now unique, in the ACP-encoding region. The fusion gene can be isolated together with the P-mel promoter located in front, as BamHI-BamHI cassette from plasmid p4.

1.4 Construction of a Shuttle Plasmid and Assembly to Give a Basic Plasmid for the MCA Method

The basic plasmid is to be composed of the E. coli plasmid pSP72 (Promega) and the streptomyces plasmid pIJ702 [24]. The cloning steps necessary for this are carried out in E. coli. FIG. 11 summarizes the cloning products obtained. Firstly, the two plasmids are cloned with PstI and BglII, and the PstI-BglII fragment (562 bp) containing the melanin promoter (P-mel) from pIJ702 is separated by agarose gel electrophoresis. The two truncated plasmids are then assembled to result in plasmid pX.

A BamHI cleavage site present in pIJ702 need not necessarily be deleted because the basic plasmid which results later must be cut only with RS2 (BglII) and RS3 (EcoRI) in order to insert new RS-1-3 elements. However, in order to obtain a universal starting plasmid for the construction of other basic plasmids (which are, however, not detailed in this example), the BamHI cleavage site is deleted by PCR. For this Purpose, the region from the PstI cleavage site up to the BamHI cleavage site is amplified from plasmid pX using the primers 5′-CAGCTGAAGCTTGCATGCCTGCAGCCGGG-3′ and 5′-GCAACGAAGATCTGGCGGCCGTGGGCGAA-3′. The PstI cleavage site is retained in the resulting>762 bp PCR product, but the BamHI cleavage site is mutagenized to a BglII cleavage site. The PCR product is therefore cut with PstI and BglII, and the corresponding region in plasmid pX (from PstI to BamHI) is replaced by the PCR product. Fusion of the BglII and BamHI cleavage sites results in a sequence which cannot be cleaved by either of the restriction enzymes. This replacement results in plasmid pxmut which has a unique BglII cleavage site for insertion of gene cassettes.

Correspondingly, the gene cassette produced previously in plasmid p4 and coding for the actinomycin starter module and the TE domain with the P-mel promoter in front is isolated as BamHI-BamHI cassette from plasmid p4 and cloned into the BglII cleavage site of plasmid pxmut. Fusion of the BamHI and BglII cleavage sites destroys them and results in the complete basic plasmid pBASIS which has a unique RS2 (BglII) and unique RS3 (EcoRI) cleavage site, which fix the RS-2-3 linker region.

### Example 2

### Description of Various Mutagenesis Methods for Deleting Restriction Cleavage Sites and the Exemplary Procedure for a Double Mutagenesis Using One of the Described Methods

Since the establishment of targeted mutagenesis with single-stranded DNA based on M13 mutagenesis [20], numerous other methods permitting efficient and direct mutagenesis with double-stranded DNA, such as, for example, with plasmids, have been developed. Even if the details of the individual methods differ, the actual mutagenesis is always based on formation of hybrid between a DNA strand of the DNA fragment to be mutagenized and a DNA oligomer (mutagenesis primer) which is about. 10-30 base pairs long and whose sequence is non-complementary only at the site to be mutagenized. To form the hybrid, the double-stranded DNA to be mutagenized is denatured, such as, for example, by alkaline denaturation. One of the two resulting DNA single strands can then form a hybrid with the mutagenesis primer. The 3′ end of the mutagenesis primer is extended after the formation of the hybrid, using the original DNA fragment as template. This extension usually takes place in vitro through the use of a DNA polymerase. In the case of circular DNA templates, such as plasmid single strands, the newly produced DNA strand can be closed in vitro by subsequent use of a DNA ligase. In this way, a double-stranded DNA plasmid in which one of the two strands contains the mutagenesis sequence is produced again. Selection for the mutagenesis sequence and deletion of the original sequence depends on the method used. For example, in vitro DNA synthesis can be carried out, after formation of the hybrid with the mutagenesis primer, using the nucleotide dCTPxS in place of dCTP [18, 19], whereby the newly synthesized strand is protected from cleavage with the enzyme NciI. The strand with the original sequence is, by contrast, cleaved by NciI and subsequently degraded, completely or substantially, with the enzyme exonuclease III. The template then used for renewed in vitro. DNA synthesis is the strand with the mutagenesis sequence, resulting in a double-stranded plasmid, both of whose strands contain the mutagenesis sequence. In other methods, the selection for the mutagenesis sequence takes place in vivo. This entails the plasmid obtained after the in vitro synthesis with the mutagenesis primer being directly transformed into E. coli. Plasmid replication in E. coli then results in principle in two sorts of double-stranded plasmids, plasmids with the original sequence and plasmids with the mutagenesis sequence. These methods generally use, in the in vitro DNA synthesis which has taken place beforehand, not only the mutagenesis primer but at the same time also one or more additional primers which delete a unique restriction cleavage site in the original plasmid. [27] or restore a defective resistance gene in the original plasmid. This makes it possible for a subsequent selection then to take place through the use of the unique restriction enzyme, or selection can be for the acquired resistance.

Synthesis of the mutagenesis primers required in all the methods is available from numerous companies. Moreover, numerous kits containing all the other components required, such as plasmids and E. coli strains, are obtainable for carrying out the targeted mutagenesis. The use of such kits is now standard in molecular biology. The mutagenesis for deletion of the BglII and BamHI cleavage site from the DNA fragment coding for the TE domain of ACMS III is carried out using the kit “Altered Sites® II in vitro Mutagenesis System” from Promega, but the mutagenesis is also possible in principle using other methods. The described kit uses a mutagenesis plasmid (pALTER1) which harbors the genes of two selection markers (ampicillin=Amp; tetracycline=Tet) and a polylinker for insertion of the DNA fragment to be mutagenized, in this case the EcoRI-PstI fragment generated by PCR and coding for the TE domain of ACMS III. One of the two resistance genes in pALTER1 harbors a mutation, resulting in this gene being unable to confer resistance (S phenotype). The other resistance gene is, by contrast, intact and confers an antibiotic resistance (R phenotype). During the DNA synthesis carried out in vitro with the mutagenesis primer, the original intact resistance gene is inactivated (R to S) and the originally defect resistance gene is restored (S to R) by addition of two further primers besides the mutagenesis primer. Both primers required are part of the leit. The plasmid obtained by in vitro synthesis is transformed into E. coli and selected for the newly acquired resistance and for the loss of the original resistance. It is possible in this way to introduce a plurality of mutations successively into the DNA fragment inserted into pALTER1 by alternately destroying or restoring the resistance genes on pALTER1. The BamHI cleavage site in the inserted EcoRI-PstI fragment is deleted by using the mutagenesis primer 5′-GAGATCGGCCGCATCCTGTCGGCCA-3′, the primer for conversion of AmpS to AmpR, and the primer for conversion of TetR to TetS. After ampicillin selection, the loss of tetracycline resistance is also checked by replica plating of the E. coli transformants. The plasmid is isolated from one of these transformants and also checked for loss of the BamHI cleavage site by in vitro restriction with BamHI. The BglII cleavage site remaining in the EcoRI-PstI fragment is deleted by using the mutagenesis primer 5′-ACACGATCACCGAAATCTCGGCCAAC-3′, the primer for conversion of AmpR to AmpS, and the primer for conversion of TetS to TeTR. After tetracycline selection, loss of ampicillin resistance is checked by replica plating of the E. coli transformants. The plasmid is isolated from one of these transformants and also checked for loss of the BglII cleavage site by in vitro restriction with BglII. It is then possible to isolate the mutagenized EcoRI-PstI fragment again from the resulting plasmid. The introduced mutations have destroyed the BamHI and BglII cleavage sites, but the mutagenized reading frame codes for the same protein sequence as the original EcoRI-PstI fragment.

### Example 3

### Synthesis of an RS-1-3 Element from an NRPS System for Use with Basic Plasmid pBASIS

The basic plasmid PBASIS prepared in example 1 has an RS-2-3 linker within the ACP-encoding region, which makes it possible to insert RS-1-3 elements appropriate for this in the. MCA method. The RS-1-3 element to be used for insertion is the region which codes for a repetitive NRPS unit, and which makes the activation of the substrate amino acid glycine possible, from the ACMS III gene (acmC) (mod 4 in FIG. 2). To simplify the necessary modifications, this region is first isolated as 4.8 kb NotI-NotI fragment from the acmC gene (from cosmid pP1, [26]) and subcloned into the NotI cleavage site of pSL1180 (Pharmacia), resulting in plasmid pMEGLY. The NotI cleavage site in the 5′ region of the acmC gene section cloned into pMEGLY is located in the coding region of the ACP domain of the preceding module (mod 3), and the NotI cleavage site in the 3′ region of the gene section is already behind the region coding for mod 4:

```
acmC sequence (mod 4) in S. chrysomallus:
3097 4430 6407 7522 7879
: : : : :
GCGGCCGCCGTCGCCGCGCAC.TGGATCC.CGGATCC..GCCGCGGTGGCCGCCCGG..GCGGCCGC
A A A V A A H W I R I A A V A A R
    NotI BamHI BamHI NotI
      ------------------ ------------------
        RS-2-3 linker region RS-2-3 linker region
          (in ACP mod 3) (in ACP mod 4)
acmC sequence (mod 4) in plasmid pMEGLY:
          3097 4430 6407 7622 7879
          : : : : :
ctgcagGCGGCCGCCGTCGCCGCGCAC.TGGATCC.CGGATCC..GCCGCGGTGGCCGCCCGG..GCGGCCGC..tctaga
            A A A V A A M W I R I A A V A A R
PstI NotI BamHI BamHI NotI XbaI
                  ------------------ ------------------
                    RS-2-3 linker region RS-2-3 linker region
                      (in ACP mod 3) (in ACP mod 4)
```

In the depicted orientation, the acmC fragment cloned into pMEGLY acquires a PstI cleavage site introduced by the pSL1180 polylinker in the 5′ region, and an XbaI cleavage site in the 3′ region (sequence in small letters). The numbering of the base pairs relates to the original ATG start codon of the acmC gene, and the encoded amino acid sequence is indicated underneath the DNA sequence. For mutagenesis of the two internal BamHI cleavage sites (position 4430 and 6407 in acmC), the fragment is isolated as PstI-XbaI fragment from pMEGLY and cloned into the mutagenesis plasmid pALTER. 1 (PstI+XbaI). The mutagenesis itself is carried out as described in example 2 in the form of a double mutagenesis, using the mutagenesis primers 5′-GTCGAGCAGGTGTATCCAGCGGTTG-3′ and 5′-CGGGCGCGAGGATGCGCAGGGCGTGGT-3′ for the mutagenesis. This mutagenizes the BamHI cleavage sites at position 4430 to 5′-GGATAC-3′ and at position 6407 to 5′-GCATCC-3′, but the amino acid sequence encoded by the fragment is not changed. A subsequent PCR with the primers 5′-GGCGGATCCGTCGCCGCGCACCTCGACCT-3′ and 5′-CAGGAATTCGGCCACAGATCTCGGGGTCGGGCCCTCGACAGCGAGC-3′ generates the cleavage sites RS1=BamH1, RS2=BglI1 and RS3=EcoRI at the appropriate positions in the RS-2-3 linker regions, resulting in the finished RS-1-3 element.

AcmC sequence (mod 4) as RS-1-3 element:
3100 4430 8407 7522 7539
: : : : :
GGATCCGTCGCCGCGCAC.TGGATAC.CGCATCC..AGATCTGTGGCCGAATTC
R S V A A H W I R I R S V A E F
BamHI BglII EcoRI
------------------ ------------------
RS1 RS2 RS3

Cloning of the RS-1-3 element into plasmid pTZ18 (BamHI+EcoRI) results in the element plasmid PELEMENT in which the RS-1-3 element can be replicated and further modified.

### Example 4

### Cyclic Insertion of RS-1-3 Elements into the Basic Plasmid pBASIS

All the steps to be carried out for the cyclic insertion of RS-1-3 elements into basic plasmids are based on standard methods of molecular biology, and reference is made in the following only to important features in the application thereof. The recombination-defective E. coli strain DH5α is used for cloning. E. coli strains with defects in the recombination system increase the stability of repetitive DNA elements during cloning and are therefore preferably employed in cloning steps in the MCA method. For insertion of RS-1-3 elements which have been tailored as described in example 3 to the basic plasmid pBASIS prepared in example 1, firstly pBASIS is isolated from E. coli.

Isolation of the plasmid DNA can in principle be carried out by all conventional preparation methods, but the simple method of alkaline lysis [28] with subsequent ethanol precipitation is very suitable because of its DNA-sparing properties and simple procedure. This method is therefore used for isolation of pBASIS and for later isolation of pBASIS derivatives. The isolated basic plasmid pBASIS is subsequently cut with the restriction enzymes R2 (BglII) and R3 (EcoRI). Restriction of the DNA must be carried out under the optimal reaction conditions for the respective enzymes R2 and R3. If the buffer systems are incompatible or if the incubation temperatures for the enzymes differ it is therefore necessary for the restriction to take place in two consecutive steps. However, a common buffer can be used for restriction with BglII and EcoRI (GibcoBRL) at 37° C., and a reaction time of 6-12 hours with at least two units of each enzyme per μg of DNA is sufficient.

The RS-2-3 linker which has been cut out, and the restriction enzymes used, can be removed by simple agarose gel electrophoresis. Ethidium bromide is added to the agarose, making the DNA visible on irradiation with UV light. The DNA of the linearized basic plasmid is then, entrapped in agarose, prepared from the agarose gel after the electrophoresis. In a random approach, in which a DNA mixture of basic plasmids differing in size is present after cutting with R2+R3, the DNA mixture, entrapped in agarose, is prepared from the agarose gel correspondingly. Only a short migration distance is generally necessary in the electrophoresis because the electrophoresis serves for removal of the RS-2-3 linker, not for fractionation of the basic plasmids. The RS-2-3 linker resulting on use of pBASIS from example 0.1 has a size of 0.018 kb in all plasmids derived from pBASIS, whereas the linearized basic plasmids are always larger than 10 kb. Thus, on use of a 1% agarose gel, a separation distance in which a DNA reference fragment of 5 kb migrates a distance of 2-3 cm is sufficient. A short migration distance is to be preferred in particular for random approaches because in this case there is negligible fractionation of basic plasmids larger than 10 kb, and the prepared DNA mixture is therefore surrounded by only a small amount of agarose. It is in turn possible to use various methods for detaching the DNA from the agarose, such as, for example, electroelution or melting of the agarose on use of low melting temperature agarose. Care must be taken in the choice of the method that the DNA is removed from the agarose without shear forces if possible. The method used for purifying PBASIS is therefore very gentle. The prepared agarose block is mixed with about 15 times the volume of phenol (equilibrated with 10 mM Tris HCl, pH 8), frozen at −80° C. for 40 minutes and then, still frozen, centrifuged in a commercial bench centrifuge at room temperature and 10 000 rpm for 40 minutes. After repeated freezing and renewed centrifugation, the DNA can be isolated from the aqueous phase by customary ethanol or isopropanol precipitation. The basic plasmid prepared in this way can be employed directly for ligation with an RS-1-3 element. The RS-1-3 element to be used is obtained by cutting the appropriate element plasmid with the enzymes R1+R3 and then removing the RS-1-3 element from the remaining plasmid portion by agarose gel electrophoresis and subsequently purifying it. The requirements applying to the restriction with R1+R3 in relation to the optimal restriction conditions are the same as for the restriction with R2+R3 already described. When preparing the RS-1-3 element from the element plasmid pELEMENT prepared in example 3, the restriction with BamHI and EcoRI (GibcoBRL) can again be carried out jointly at 37° C. Before the ligation, the resulting RS-1-3 element is dephosphorylated in order to avoid subsequent multiple integration due to concatemer formation. For this purpose, the DNA can be incubated with SAP (shrimp alkaline phosphatase, Amersham) at 37° C. for 1 h, and the SAP can then be deactivated at 65° C. for 30 min. Ligation of pBASIS (BglII-EcoRI) with the dephosphorylated RS-1-3 element (BamHI-EcoRI) takes place under the usual conditions with T4 DNA ligase at −16° C. with an approximately three-fold. Excess of RS-1-3 elements for 10-16 hours. The ligation mixture is then transformed into E. coli, and the newly produced basic plasmid, with inserted RS-1-3 element, is isolated from one of the resulting transformants. In a random approach in which various novel basic plasmids are produced, either the plasmid isolation takes place in parallel from a plurality of different transformants, or the various plasmids are isolated together from a transformant mixture which is obtained, for example, by rinsing off the transformation plate. A new RS-1-3 element can then be inserted again into each new basic plasmid by repeating the procedure described above. For gene expression, the new basic plasmid is then transformed for example into a streptomyces strain. The strain S. lividans TK64, for example, is suitable for expression of new NRPS genes with subsequent formation of catalytically active enzymes [4, 26].

### CITATION

(R1)

Konz, D. and Marahiel, M. A. (1999) How do peptide synthetases generate structural diversity. ? Chemistry & Biology 6:R39-R48.

(R2)

Hopwood, D. A. (1997). Genetic Contributions to Understanding Polyketide Synthases. Chem. Rev. 0.97, 2465-2497.

(R3)

Cane, D. E. and Walsh, C. T. (1999). The parallel and convergent universes of polyketide synthases and nonribosomal peptide synthetases. Chemistry & Biology. 6, No. 12, R319-R325.

(1)

Stachelhaus, T., Schneider, A. and Marahiel, M. A. (1995). Rational design of peptide antibiotics by targeted replacement of bacterial and fungal domains. Science 269, 69-72.

(2)

deFerra, F., Rodriguez, F., Tortora, O., Tosi, C. and Grandi, G. (1997). Engineering of Peptide Synthetases. JBC 272, No. 40, 25304-25309.

(3)

Schneider, A., Stachelhaus, T. and Marahiel, M. A. (1998). Targeted alteration of the substrate specificity of peptide synthetases by rational module swapping. Mol. Gen. Genet. 257, 308-318.

(4)

Schauwecker, F., Pfennig, F., Grammel, N. and Keller, U. (2000). Construction and in vitro analysis of a new bi-modular polypeptide synthetase for synthesis of N-methylated acyl peptides. Chem. & Biol. 7, 287-297.

(5)

Cortes, J., Wiesmann, K. E., Roberts, G. A., Brown, M. J., Staunton, J. and Leadlay, P. F. (1995). Repositioning of a domain in a modular polyketide synthase to promote specific chain cleavage. Science. 268(5216):1487-1489.

(6)

Kuhstoss, S., Huber, M., Turner, J. R., Paschal, J. W. and Rao, R. N. (1996). Production of a novel polyketide through the construction of a hybrid polyketide synthase. Gene 183(1-2):231-236.

(7)

McDaniel, R., Kao, C. M., Hwang, S. J. and Khosla, C. (1997). Engineered intermodular and intramodular polyketide synthase fusions. Chem Biol. 4(9):667-674.

(8)

Ruan, X., Pereda, A., Stassi, D. L., Zeidner, D., Summers, R. G., Jackson, M., Shivakumar, A., Kakavas, S., Staver, M. J., Donadio, S. and Katz, L. (1997). Acyltransferase domain substitutions in erythromycin polyketide synthase yield novel erythromycin derivatives. 179(20):6416-6425.

9)

Staunton, J. (1998). Combinatorial biosynthesis of erythromycin and complex polyketides. Current Opinion in Chemical Biology 2, 339-345.

(10)

Gokhale, R. S., Tsuji, S. Y., Cane, D. E. and Koshla, C. (1999). Dissecting and Exploiting Intermodular Communication in Polyketidee Synthases. Science 284, 482-485.

(11)

Ranganathan, A., Timoney, M., Bycroft, M., Cortes, J., Thomas, I. P., Wilkinson, B., Kellenberger, L., Hanefeld, U., Galloway, I. S., Staunton, J. and Leadlay, P. F. (1999). Knowledge-based design of bimodular and trimodular polyketide synthases based on domain and module swaps: a route to simple statin analogues. Chemistry & Biology 6, 731-741.

(12)

Methods for generating and screening novel metabolic pathways.

U.S. Pat. No. 5,824,485

U.S. Pat. No. 5,783,431

(13)

Saiki, R. K., Scharf, S., Faloona, F., Mullis, K. B., Horn, G. T., Erlich, H. A. and Arnheim, N. (1985). Enzymatic amplification of beta-globin genomic sequences and restriction site analysis for diagnosis of sickle cell anemia. Science 230, 1350-1354.

(14)

Mullis, K, Faloona, F., Scharf, S., Saiki, R., Horn, G. and Erlich H. (1986). Specific enzymatic amplification of DNA in vitro: the polymerase chain reaction. Cold Spring Harbour Symposia on Quantitative Biology, 51, 263-273.

(15)

Mullis, K. B. and Faloona, F A. (1987). Specific synthesis of DNA in vitro via a polymerase-catalyzed chain reaction. Methods Enzymol. 155:335-350.

(16)

Landt, O., Grunert, H. P. and Hahn, U. (1990). A general method for rapid site-directed mutagenesis using the polymerase chain reaction. Gene. 96(1):125-128.

(17)

Sambrook, J., Fritsch, E. F. and Maniatis, T. (1989). Molecular Cloning: A Laboratory Manual, 2nd ed. Cold Spring Harbor Laboratory Press, Cold Spring Harbor, N.Y.

(18)

Taylor, J. W., Ott, J. and Eckstein, F. (1985). The rapid generation of oligonucleotide-directed mutations at high frequency using phosphorothioate-modified DNA. Nucleic Acids Res. 13(24):8765-8785.

(19)

Nakamaye, K. L. and Eckstein, F. (1986). Inhibition of restriction endonuclease Nci I cleavage by phosphorothioate groups and its application to oligonucleotide-directed mutagenesis. Nucleic Acids Res.14(24):9679-9698.

(20)

Kunkel, T A., Roberts, J. D. and Zakour, R. A. (1987). Rapid and efficient site-specific mutagenesis without phenotypic selection. Methods Enzymol. 154:367-382.

(21)

Muth, G., Nussbaumer, B., Wohlleben, W. and Puhler, A. (1989). A vector system with temperature-sensitive replication for gene disruption and mutational cloning in Streptomyces. Mol. Gen. Genet. 219, 341-348.

(22)

Vara, J., Lewandowska-Skarbek, M., Wang, Y. G., Donadio, S. and Hutchinson, C. R. (1989). Cloning of genes governing the deoxysugar portion of the erythromycin biosynthesis pathway in Saccharopolyspora erythraea (Streptomyces erythreus). J Bacteriol. 171(11):5872-5881.

(23)

Quiros, L. M., Aguirrezabalaga, I., Olano, C., Mendez, C. and Salas, J. A. (1998). Two glycosyltransferases and a glycosidase are involved in oleandomycin modification during its biosynthesis by Streptomyces antibioticus. Mol. Microbiol. 28(6),1177-1185.

(24)

Katz, E., Thompson, C. J. and Hopwood, D. A. (1983). Cloning and Expression of the tyrosinase gene from Streptomyces antibioticus in Streptomyces lividans. J. Gen. Microbiol. 129, 2703-2714.

(25)

Hopwood, D. A., Bibb, M. J., Chater, K. F., Kieser, T., Bruton, C. J., Kieser, by M., Lydiate, D. J., Smith, C. P., Ward, J. M. and Schrempf, H. (1985). Genetic manipulation of Streptomyces. A laboratory manual. The John Innes Foundation, Norwich.

(26)

Schauwecker, F., Pfennig, F., Schroder, W. and Keller, U. (1998). Molecular cloning of the actinomycin synthetase gene cluster from Streptomyces chrysomallus and functional heterologous expression of the gene encoding actinomycin synthetase II. J. Bacteriol. 180(9):2468-2474.

(27)

Deng, W. P. and Nickoloff, J. A. (1992). Site-directed mutagenesis of virtually any plasmid by eliminating a unique site. Anal. Biochem.200(1):81-88.

(28)

Birnboim, H. C. and Doly, J. (1979). A rapid alkaline extraction procedure for screening recombinant plasmid DNA. Nucleic Acids Res. 7(6):1513-23.

1. A method for preparing a DNA coding for a polypeptide composed of a plurality of sections in a circular DNA vector, which comprises the following steps:
a. restriction of unique restriction cleavage sites RS1 and RS3 or of unique restriction cleavage sites RS2 and RS3 of the DNA vector;
b. ligation of a DNA fragment which codes for at least one section of the polypeptide into the DNA vector obtained in (a),
i. where one end of the DNA fragment has been obtained by restriction of a restriction cleavage site RS1 and the second end of the DNA fragment has been obtained by restriction of a restriction cleavage site RS3, and the DNA fragment has no internal restriction cleavage site RS1 or internal restriction cleavage site RS3,
ii. where the restriction cleavage site RS1 is located at the 5′ end of the region (AKB) coding for the at least one section, and the restriction cleavage site RS3 is located at the 3′ end of the AKB,
iii. where the DNA fragment has a unique restriction cleavage site RS2 which is located between the restriction cleavage sites RS1 and RS3, and −iv. where the ends generated by restriction of the restriction cleavage sites RS1 and RS2 are compatible with one another, the DNA sequence resulting from the ligation is different from the restriction cleavage sites RS2 and RS3, and the ends generated by restriction of the restriction cleavage sites RS2 and RS3 are not compatible with one another;
c. restriction of the unique restriction cleavage sites RS2 and RS3 in the DNA vector obtained in (b);
d. ligation of another DNA fragment which codes for at least one other section of the polypeptide into the DNA vector obtained in (c), where the AKBs form a continuous reading frame and where the conditions defined in b) i.) to iv.) are to be applied appropriately, and, if desired,
e. at least one repetition of steps c) and (d).

2. The method as. Claimed in claim 1, where the DNA which codes for at least one section and which is employed in stage (d) as second or later DNA fragment has been obtained by restriction of restriction cleavage sites RS2 and RS3, and the DNA fragment has no internal restriction cleavage site RS2 or internal restriction cleavage site RS3, where the restriction cleavage site RS2 is located at the 5′ end of the AKB and the restriction cleavage site RS3 is located at the 3′ end of the AKB, and this DNA fragment is thus employed as the fragment concluding the cycle.

3. The method as claimed in claim 1 or 2, where the DNA coding for at least one section has at least one mutation compared with the naturally occurring nucleic acid sequence.

4. The method as claimed in any of the preceding claims, where the DNA vector comprises a protein-encoding reading frame which is extended by at least one AKB in the same reading frame.

5. The method as claimed in claim 4, where the protein-encoding reading frame comprises at least one AKB.

6. The method as claimed in any of the preceding claims, where the DNA vector is a plasmid vector, a lambda vector, a cosmid vector, the replicative form of the genome of a filamentous phage, or an artificial chromosome.

7. The method as claimed in claim 6, where the plasmid vector is a plasmid which stably replicates in at least one bacterium, preferably in Escherichia coli and/or in at least one streptomyces, and which has a selection marker and a cloning site comprising at least the unique restriction cleavage sites RS1 and. RS3 or the unique restriction cleavage sites RS2 and RS3.

8. The method as claimed in any of the preceding claims, where the DNA fragments have been obtained by PCR amplification from the genome of at least one microorganism and/or at least one plant, preferably from the genome of actinomyces, and the restriction cleavage sites RS1, RS2 and RS3 have been introduced by means of directed mutagenesis.

9. The method as claimed in any of the preceding claims, where a mixture of at least two DNA fragments which comprise different AKBs is employed in step (b) and/or (d).

10. A method for preparing polypeptides consisting of at least two sections,
a. where the at least one DNA present according to the method as claimed in any of claims 1-9 in the DNA vector and obtained therefrom by restriction is cloned into an expression vector,
b. where the expression vector has a promoter and a start codon and termination codon for the reading frame of the at least one DNA which codes for a polypeptide consisting of at least two sections,
c. where the resulting expression vector is introduced into a host cell for expression of the cloned at least one DNA,
d. where the expression vector undergoes stable autonomous replication in the host cell or is stably integrated into the genome of the host cell, and
e. where the at least one DNA is expressed in the host cell and, if desired, the at least one expression product is isolated from the host cell.

11. A method for preparing polypeptides consisting of at least two sections,
a. where the at least one vector containing the DNA is introduced by the method as claimed in any of claims 1-9,
b. where the at least one DNA vector has a promoter and a start codon and termination codon for the reading frame of the DNA sequence which codes for a polypeptide consisting of at least two sections,
c. where the at least one DNA vector undergoes stable autonomous replication in the host cell or is stably integrated into the genome of the host cell, and
d. where the DNA is expressed in the host cell and, if desired, the at least one expression product is isolated from the host cell.

12. The method as claimed in claim 10 or 11, where the host cell is a microorganism, preferably a bacterium of the genus Streptomyces, Bacillus or Escherichia.
